(19) United States
(12) Patent Application Publication
(10) Pub. No.: US 2025/0230425 A1
(43) Pub. Date: Jul. 17, 2025

(54) MATERIALS AND METHODS FOR CREATING STRAINS OF SACCHAROMYCES CEREVISIAE THAT EXHIBIT AN INCREASED ABILITY TO FERMENT OLIGOSACCHARIDES INTO ETHANOL

(71) Applicant: Xylogenics, Inc., Indianapolis, IN (US)

(72) Inventors: Mark Goebl, Indianapolis, IN (US); Joshua Heyen, Brownsburg, IN (US); Nadaraj Palaniappan, Carmel, IN (US); Ross Cocklin, Keene, NH (US); Kathryn Houin, Lebanon, IN (US); Matthew Kelker, Zionsville, IN (US)

(21) Appl. No.: 18/969,650

(22) Filed: Dec. 5, 2024

Related U.S. Application Data
(63) Continuation of application No. 17/261,454, filed on Jan. 19, 2021, now Pat. No. 12,203,111.
(60) Provisional application No. 62/700,679, filed on Jul. 19, 2018.

(51) Int. Cl. C12N 9/24; C12N 15/52

(52) U.S. Cl. CPC C12N 9/2402; C12N 15/52; C12Y 302/0102

(57) ABSTRACT

A recombinant yeast strain is disclosed. The strain comprises a strain of S. cerevisiae and an exogenous MAL1 gene cluster and an exogenous MAL2-8c gene; the MAL1 gene cluster with at least 80 percent sequence identity to SEQ ID NO: 2; the MAL2-8c gene with at least 80 percent sequence identity to SEQ ID NO: 3. The strain is capable of fermenting high maltose syrups into ethanol in the presence of glucose. The presence of glucose may prevent robust maltose fermentation; the strain enhances co-fermentation of maltose and glucose. Maltose may comprise a weight less than 4.64 percent of the total volume after 24 hours of fermentation in corn mash with 31.3 percent solids treated with a 1 percent solution of maltogenic alpha amylase. Ethanol may comprise a weight of at least 4.16 percent of total volume after no more than 15 hours of fermentation in corn mash with 32.6 percent solids treated with 0.07 percent glucoamylase.

### PRIORITY CLAIM

The present application is a continuation of U.S. patent application Ser. No. 17/261,454 titled “MATERIALS AND METHODS FOR CREATING STRAINS OF SACCHAROMYCES CEREVISIAE THAT EXHIBIT AN INCREASED ABILITY TO FERMENT OLIGOSACCHARIDES INTO ETHANOL” filed on Jul. 19, 2021, which is a national phase filing of PCT/International Patent Application No. PCT/US2019/042605 titled “MATERIALS AND METHODS FOR CREATING STRAINS OF SACCHAROMYCES CEREVISIAE THAT EXHIBIT AN INCREASED ABILITY TO FERMENT OLIGOSACCHARIDES INTO ETHANOL”, filed on Jul. 19, 2019, which claims priority to U.S. Provisional Patent Application No. 62/700,679 titled “MATERIALS AND METHODS FOR CREATING STRAINS OF SACCHAROMYCES CEREVISIAE THAT EXHIBIT AN INCREASED ABILITY TO FERMENT OLIGOSACCHARIDES INTO ETHANOL”, filed on Jul. 19, 2018.

The present application claims priority to and incorporates by reference in full the following applications: (a) U.S. patent application Ser. No. 17/261,454 titled “MATERIALS AND METHODS FOR CREATING STRAINS OF SACCHAROMYCES CEREVISIAE THAT EXHIBIT AN INCREASED ABILITY TO FERMENT OLIGOSACCHARIDES INTO ETHANOL” filed on Jul. 19, 2021; (b) PCT/International Patent Application No. PCT/US2019/042605 titled “MATERIALS AND METHODS FOR CREATING STRAINS OF SACCHAROMYCES CEREVISIAE THAT EXHIBIT AN INCREASED ABILITY TO FERMENT OLIGOSACCHARIDES INTO ETHANOL”, filed on Jul. 19, 2019; (c) U.S. Provisional Patent Application No. 62/700,679 titled “MATERIALS AND METHODS FOR CREATING STRAINS OF SACCHAROMYCES CEREVISIAE THAT EXHIBIT AN INCREASED ABILITY TO FERMENT OLIGOSACCHARIDES INTO ETHANOL”, filed on Jul. 19, 2018.

### CROSS-REFERENCE TO RELATED APPLICATIONS

The present application is related to and incorporates by reference in full the following applications: (a) U.S. patent application Ser. No. 17/261,454 titled “MATERIALS AND METHODS FOR CREATING STRAINS OF SACCHAROMYCES CEREVISIAE THAT EXHIBIT AN INCREASED ABILITY TO FERMENT OLIGOSACCHARIDES INTO ETHANOL” filed on Jul. 19, 2021; (b) PCT/International Patent Application No. PCT/US2019/042605 titled “MATERIALS AND METHODS FOR CREATING STRAINS OF SACCHAROMYCES CEREVISIAE THAT EXHIBIT AN INCREASED ABILITY TO FERMENT OLIGOSACCHARIDES INTO ETHANOL”, filed on Jul. 19, 2019; (c) U.S. Provisional Patent Application No. 62/700,679 titled “MATERIALS AND METHODS FOR CREATING STRAINS OF SACCHAROMYCES CEREVISIAE THAT EXHIBIT AN INCREASED ABILITY TO FERMENT OLIGOSACCHARIDES INTO ETHANOL”, filed on Jul. 19, 2018.

### REFERENCE TO SEQUENCE LISTING

This application contains a Sequence Listing submitted via EFS-wed which is hereby incorporated by reference in its entirety for all purposes. The ASCII copy, created on Jul. 18, 2019, is named XYLO_0002_01_WO_ST25.txt and is 43KB in size.

### FIELD

Aspects of the invention relate to making and using strains of Saccharomyces cerevisiae that are capable of efficiently fermenting high maltose syrups into ethanol thereby either eliminating or reducing the need to convert disaccharides and trisaccharides into glucose through the addition of glucoamylase enzymes to yeast feed stocks.

### BACKGROUND

Various species of Saccharomyces are among the most important industrially grown microorganisms. Long used to leaven bread, produce beer and wine, and as a source of food flavorings and micronutrients, these organisms now play a central role in the production of fuel, facilitating the conversion of sugars to ethanol. A metabolically complex organism, yeast can grow both aerobically and anaerobically as well, if certain nutritional conditions are met. When grown commercially, as in the production of yeast used to support the commercial baking industry, yeasts such as Saccharomyces cerevisiae are grown in highly aerated fermentation tanks. The growth of yeast under these conditions is manipulated to favor the production of yeast biomass. One way in which this is accomplished is to schedule the addition of sugars, such as D-glucose, and the rate of oxygen transfer to the yeast to encourage aerobic growth. Various strains of Saccharomyces can also be grown under conditions designed to maximize the production of ethanol. Oftentimes, when the object is to maximize the conversion of sugar to ethanol, the level of oxygen in the fermentation vessel is reduced relative to the levels of oxygen used in the vessel during yeast biomass production in order to favor anaerobic growth.

Most strains of Saccharomyces prefer growth on D-glucose although many strains are known to grow on other naturally occurring hexoses and even some disaccharides as well. The ability of different species of Saccharomyces to grow on different sugars and in the presence of different levels of oxygen accounts for much of its commercial utility including the central role that yeast currently plays in the conversion of plant bio-mass into ethanol for various uses including its use as a fuel.

One of the best-known pathways for the production of ethanol by yeast is the fermentation of 6-carbon sugars (hexoses) into ethanol, especially D-glucose. One widely used feedstock for the production of ethanol is the polysaccharide starch. Starch is a simple polymer consisting of chains of D-glucose. Currently, in the United States at least, starch derived from corn kernels is the preferred feed stock for bio-ethanol production by Saccharomyces cerevisiae.

A single kernel of corn is comprised of ˜65-80 percent starch depending on the growing season and the specific corn variety. Starch in its most basic form is a polymer of many glucose molecules linked through glycosidic bonds. This polymer can take on two basic forms. Amylose is primarily a linear glucose polymer that can contain up to 600 glucose molecules (known as DP or degree of polymerization) linked together by α-(1,4) linkages. Amylopectin however consists of large highly branched glucose polymers that can range in degree of polymerization from hundreds of thousands to millions of glucose units. Glucose units in amylopectin are linked together by both α-(1,4) and α-(1,6) linkages with the latter type providing the branching structure. Together, many amylose and amylopectin molecules intertwine into an ordered superstructure known as a starch granule (looks much like a very small onion with concentric layers). A single kernel of corn contains many starch granules consisting of 70-80 percent amylopectin and 20-30 percent amylose.

Starch granules serve to store chemical energy for the seed in a very compact and recalcitrant state. This allows for a large amount of energy to be packed into a small space while inhibiting the use of this energy reserve by microbes. In this form, starch is unavailable to the cells of the seed for energy and must therefore be broken down by enzymes into metabolizable molecules (monosaccharide and disaccharide sugars, i.e. glucose and maltose). The initial steps in producing fuel ethanol from corn are designed to achieve the same goal; breakdown of corn starch to usable cellular energy. However, the cellular energy is being used for fermentation by yeast and converted into ethanol.

The process to extract and hydrolyze corn starch in preparation for yeast fermentation starts when corn is received at the ethanol production facility. Corn is received either directly from the farmer or through other intermediaries at the ethanol plant by rail or truck. Each shipment is tested for quality by monitoring percent moisture, percent foreign particles, and the presence of toxins. Each facility has its own corn standards that must be met to accept a certain corn shipment. Corn of low moisture <=20 percent, low foreign particles, and minimal toxicity enables the most efficient and highest yielding fermentations. However, corn qualities such as percentage starch content, protein content, the amylose to amylopectin ratio, as well as a multitude of other factors drastically affect fermentation yield. These factors vary by region, corn hybrid, weather, farm practices, and other unpredictable variables. It is therefore common to have drastic swings in ethanol plant productivity due to variation in the corn quality from different harvests.

Once corn has been purchased and received, it is either stored on sight or fed directly to a mill. There are two different milling procedures utilized in the United States known as wet milling or dry milling. Over 70 percent of the 13.3 Billion gallons of fuel ethanol made in the United States in 2012 was made using what is called a dry milling or dry grind process. For this reason, the application includes—dry milling although the invention disclosed herein can be used with feed stocks prepared by virtually any milling process.

The milling process includes forming the corn into fine flour using any number of milling technologies. The most common mill utilized is a hammer mill that disrupts and grinds the corn kernel using sharpened shafts (hammers) spinning at high speed around a central axis (think enclosed fan). As the hammers spin they grind corn entering from the top of the mill until the corn is ground small enough to pass through a screen of a given size. Screen size dictates the particle size of the flour and influences many downstream processes. As flour particle size rises, the downstream enzymatic hydrolysis of the starch becomes less and less complete ultimately decreasing the amount of sugar available to yeast and the amount of ethanol that can be produced from a given amount of corn. However, creating smaller particle sizes requires more work (energy) as the hammer mill must operate at a higher amperage to breakdown the particles. Smaller particle sizes also increase soluble solids in thin stillage, reducing centrifuge and evaporator efficiency during co-product feed production (Evaporation is an energy intensive process). For these reasons, milling practices vary across ethanol production facilities; on particles with an average screen sizes between 2.5 and 3 mm are utilized.

The ground corn flour is then mixed with water at a certain ratio in a slurry mixer. The ratio of water to corn flour determines the solids level of the final fermentation corn mash. The solids level is an important parameter in fuel ethanol production. This ratio ultimately determines the amount of sugar that is supplied to the yeast and therefore determines the maximum ethanol titer that can be achieved when the material is fermented. Today ethanol producers in the United States typically favor a 32 percent corn flour mixture (32 percent Solids) but solids levels can vary between 28 and 34 percent, depending on facility and season. Fermentations carried out at these solids levels are known as VHG fermentations (for Very High Gravity). The ability to carry out VHG fermentations drastically increases the efficiency of fuel ethanol production but is currently limited to the aforementioned solids levels for several reasons.

In a typical process to produce ethanol from corn the corn flour and water slurry is mixed with an α-amylase enzyme in a slurry mixer. The enzyme/corn/water mixture (mash) is then pumped to a slurry tank where it is heated to ˜90° C. to gelatinize the starch for hydrolysis by the α-amylase. The a-amylase is an endoenzyme and thus hydrolyzes glycosidic bonds within the starch granule. This action quickly reduces the viscosity of the mash as it de-polymerizes the starch polymer into shorter chain dextrins. Typically, the mash is held in the slurry tank for ˜20 minutes and is then sterilized, further gelatinized, and sheared in a jet cooker at 200° C. Jet cooked mash is then pumped into the liquefaction tanks, treated with a second dose of α-amylase, and held at 80-90° C. for two hours to further break down the starch into dextrins. The mash is then cooled to 30-34° C. and pumped into an 800,000 gallon fermentation tank along with yeast, nutrients, and a second enzyme, glucoamylase, to start a process known as SSF (Simultaneous Saccharification and Fermentation). Glucoamylase is an exo-acting β-amylase that liberates glucose from the non-reducing ends of starch polymers and dextrins. Thus, gluco-amylase ‘spoon feeds’ fermentable sugars to the yeast for fermentation to ethanol. The upstream processing required to produce fermentable sugars from starch for yeast fermentation is time and energy intensive.

Most commonly used glucoamylase enzyme technologies are designed to produce glucose from corn starch at a rate consistent with the rate that yeast will ferment glucose, which is preferred by normal yeast for fermentation. This preference is defined in part by the fact that when presented with a mixture of fermentable sugars, strains of Saccharomyces cerevisiae used to produce ethanol ferment glucose first and almost exclusively until virtually all the available glucose is fully consumed. Only after virtually all of glucose is completely consumed, will these strains of yeast switch to fermenting other sugars that may be available in the feed stock.

All the glucoamylase enzymes commonly used in the fuel ethanol industry are inhibited to various degrees by the presence of maltose; and maltose is almost always produced to some degree during the breakdown of starch. The accumulation of glucose in the fermenter is also undesirable as it increases the osmolarity of the environment in the fermentation vessel. Most strains of yeast used to produce ethanol are sensitive to the osmolarity of the fermentation environment; high osmolarity can reduce the efficiency of the fermentation and slow or even inhibit the ability to the yeast to produce ethanol. Accordingly, coordinating the rate of glucose production from the breakdown with the rate of glucose consumption by yeast is also necessitated by the need to reduce osmolality of the fermentation environment.

Because the accumulation of high concentrations of glucose in the fermenter broth may lead to stuck fermentations and tremendous yield reductions, traditional fermentation systems limit the rate of starch breakdown to coincide with the rate of yeast glucose fermentation. This limitation reduces the amount of starch that can be broken down and fermented in each 54-hour fermentation and thus limits maximum fermenter yield. Interestingly, maltose, which is also a fermentable sugar that can be produced from corn starch, is half as osmotically stressful to yeast and thus can accumulate to concentrations that are twice the acceptable glucose concentration in a fermenter. Therefore, the rate of starch breakdown can be greatly accelerated by producing the less stressful sugar maltose. Maltose production allows for higher solids to be loaded into a fermenter leading to higher ethanol titers, lower water usage, lower heat usage, and greater margins.

However, maltose fermentation in standard commercial yeast is glucose repressed and thus the efficiency of maltose fermentations is greatly inhibited by the accumulation of even small amounts of glucose in the fermenter using traditional commercial yeast. Thus, glucose repression has prevented the application of high gravity maltose fermentations. Some aspects of the present invention address the apparent difficulties of high gravity maltose fermentations.

### SUMMARY

Various strains of Saccharomyces cerevisiae are the industry standard strain for commercial production of fuel ethanol from grains such as corn. One widely used strain of S. cerevisiae is the commercially available strain Ethanol Red®. This strain has a robust system for utilizing glucose and includes a functional MAL2 locus which enables the strain to ferment maltose. Aspects of the present invention consists of a modified strain of Ethanol Red® in which maltose fermentation has been modestly improved and glucose fermentation rates have increased, thereby improving fermentation of high maltose syrups and maltose/glucose mixtures and furthermore reducing the requirement for exogenous glucoamylase enzyme. DNA sequencing and extensive genomic assembly revealed the MAL1 gene cluster in the Ethanol Red® strain to be significantly different than the MAL1 gene cluster present in many well characterized lab strains (FIG. 1 and SEQ ID NO: 1). Each MAL1 gene cluster is ˜10 Kb and encodes three genes for maltose import and breakdown. The MAL11 gene encodes a high affinity, broad specificity maltose transporter that can also transport turanose, isomaltose, alpha-methylglucoside, maltotriose, palatinose, panose, trehalose and melezitose. The MAL12 gene encodes a maltase that hydrolyzes maltose producing two glucose molecules. MAL13 encodes a transcriptional activator responsible for inducing MALI1 and MAL12 transcription in the presence of maltose. In wild type industrial and laboratory strains MAL12 and MAL13 require maltose for induction and glucose, even at a very low concentration, represses expression even in the presence of maltose. In one embodiment of the present invention, the Ethanol Red® strain was modified to also contain a functional MAL1 gene cluster which is redundant to some degree with the MAL2 cluster. The gene encoding the Mal2 transcription factor from the laboratory strain CEN.PK (SEQ ID NO: 3) was also incorporated. While this modified version of Ethanol Red® exhibited a modest increase in its ability to ferment maltose, it also exhibited a dramatic and unpredicted effect on how well it consumed glucose under a variety of commercial starch fermentation conditions. There were also robust yield improvements in the production of ethanol compared the Ethanol Red®. Furthermore, and also unexpected, the amount of exogenous glucoamylase required for complete fermentation is significantly less than what is required of other leading industrial strains.

In another embodiment, the integrated MAL1 gene cluster is not identical to SEQ ID NO: 2 but its encoded protein products share 95 percent similarity with the protein products of MAL11, MAL12 and MAL13 encoded in SEQ ID NO: 2 and shown as SEQ ID NOs: 4-6. Still other embodiments include integration of MAL1 gene cluster (SEQ ID NO: 2) and MAL2-8c gene (SEQ ID NO: 3) into other yeast strains important for ethanol production. In another embodiment, the MAL1 gene cluster and MAL2-8c genes are not integrated into the yeast genome, instead they are expressed and maintained on a plasmid. The plasmid may either be maintained at one copy per cell or as multiple copies per cell. This is dictated by the plasmid type. The plasmid may contain a CEN/ARS sequence allowing replication and faithful transmission to daughter cells. Furthermore, the MAL1 gene cluster and MAL2-8c may be expressed from the same plasmid or two separate plasmids.

A first embodiment includes a recombinant yeast strain, comprising a strain of S. cerevisiae, and an exogenous MAL1 gene cluster; wherein the strain of S. cerevisiae expresses the exogenous MAL1 gene cluster.

A second embodiment includes the recombinant yeast strain according to the first embodiment, wherein the exogenous MAL1 gene cluster is overexpressed.

A third embodiment includes the recombinant yeast strain according to any one of the first and the second embodiments, wherein the exogenous MAL1 gene cluster comprises a MAL11 gene, a MAL12 gene, and/or MAL13 gene.

A fourth embodiment includes the recombinant yeast strain according to any one of the first to the third embodiments, wherein the MAL11 gene encodes at least one agent that is involved in sugar transport; wherein the MAL12 gene encodes at least one agent that hydrolyzes maltose; and/or wherein the MAL13 gene encodes at least one agent that induces transcription of MAL11 and MAL12.

A fifth embodiment includes the recombinant yeast strain according to the fourth embodiment, wherein the at least one agent that is involved in sugar transport comprises at least one agent that transports maltose, turanose, isomaltose, alpha-methylglucoside, maltotriose, palatinose, panose, trehalose, melezitose, or any combination thereof.

A sixth embodiment includes the recombinant yeast strain according to any one of the first to the fifth embodiments, further comprising an exogenous MAL2-8c gene.

A seventh embodiment includes the recombinant yeast strain according to any one of the first to the sixth embodiments, wherein the exogenous MAL2-8c gene is overexpressed.

An eighth embodiment includes the recombinant yeast strain according to any one of the first to the seventh embodiments, wherein the recombinant strain expresses the MAL1 gene cluster and the MAL2-8c gene derived from a CEN.PK yeast strain.

A ninth embodiment includes the recombinant yeast strain according to any one of the first to the eighth embodiments, wherein the MAL1 gene cluster is integrated into the genome of the strain of S. cerevisiae.

A tenth embodiment includes the recombinant yeast strain according to any one of the first to the ninth embodiments, wherein the MAL1 gene cluster is inserted into the genome of the strain of S. cerevisiae in the subtelomeric region of chromosome VII.

An eleventh embodiment includes the recombinant yeast strain according to any one of the first to the tenth embodiments, wherein the MAL2-8c gene is integrated into the genome of the strain of S. cerevisiae.

A twelfth embodiment includes the recombinant yeast strain according to any one of the first to the eleventh embodiment, wherein the MAL2-8c gene is inserted into the genome of the strain of S. cerevisiae within a region encoding the Dubious Open Reading Frame YEL028W.

A thirteenth embodiment includes the recombinant yeast strain according to any one of the first to the twelfth embodiments, wherein the strain of S. cerevisiae is haploid, diploid, or has a ploidy number greater than two.

A fourteenth embodiment includes the recombinant yeast strain according to any one of the first to the thirteenth embodiments, wherein the MAL1 gene cluster comprises a sequence having at least 80 percent, 81 percent, 82 percent, 83 percent, 84 percent, 85 percent, 86 percent, 87 percent, 88 percent, 89 percent, 90 percent, 91 percent, 92 percent, 93 percent, 94 percent, 95 percent, 96 percent, 97 percent, 98 percent, 99 percent, and/or 100 percent homology to SEQ ID NO: 2 and the MAL2-8c gene cluster comprises a sequence having at least 80 percent, 81 percent, 82 percent, 83 percent, 84 percent, 85 percent, 86 percent, 87 percent, 88 percent, 89 percent, 90 percent, 91 percent, 92 percent, 93 percent, 94 percent, 95 percent, 96 percent, 97 percent, 98 percent, 99 percent, and/or 100 percent homology SEQ ID NO: 3.

A fifteenth embodiment includes the recombinant yeast strain according to any one of the first to the fourteenth embodiments, wherein the MAL1 gene cluster comprises a sequence having at least 85 percent homology to SEQ ID NO: 2 and the MAL2-8c gene cluster comprises a sequence having at least 85 percent homology SEQ ID NO: 3.

A sixteenth embodiment includes the recombinant yeast strain according to any one of the first to the fifteenth embodiments, wherein the MAL1 gene cluster comprises a sequence having at least 90 percent identity to SEQ ID NO: 2 and the MAL2-8c gene cluster comprises a sequence having at least 90 percent identity to SEQ ID NO: 3.

A seventeenth embodiment includes the recombinant yeast strain according to any one of the first to the sixteenth embodiments, wherein the MAL1 gene cluster comprises a sequence having at least 95 percent homology to SEQ ID NO: 2 and the MAL2-8c gene cluster comprises a sequence having at least 95 percent homology to SEQ ID NO: 3.

An eighteenth embodiment includes the recombinant yeast strain according to any one of the first to the seventeenth embodiments, wherein the MAL1 gene cluster comprises a sequence having at least 95 percent identity to SEQ ID NO: 2 and the MAL2-8c gene cluster comprises a sequence having at least 95 percent identity SEQ ID NO: 3.

A nineteenth embodiment includes the recombinant yeast strain according to any one of the first to the eighteenth embodiments, wherein the MAL1 gene cluster comprises a sequence having SEQ ID NO: 2 and the MAL2-8c gene cluster comprises a sequence having SEQ ID NO: 3.

A twentieth embodiment includes a vector comprising a MAL1 gene cluster that comprises a sequence having 80 percent, 81 percent, 82 percent, 83 percent, 84 percent, 85 percent, 86 percent, 87 percent, 88 percent, 89 percent, 90 percent, 91 percent, 92 percent, 93 percent, 94 percent, 95 percent, 96 percent, 97 percent, 98 percent, 99 percent, and/or 100 percent homology or identity to SEQ ID NO: 2.

A twenty first embodiment includes the vector according to the twentieth embodiment, further comprising a MAL2-8c gene cluster that comprises a sequence having 80 percent, 81 percent, 82 percent, 83 percent, 84 percent, 85 percent, 86 percent, 87 percent, 88 percent, 89 percent, 90 percent, 91 percent, 92 percent, 93 percent, 94 percent, 95 percent, 96 percent, 97 percent, 98 percent, 99 percent, and/or 100 percent homology or identity to SEQ ID NO: 3.

A twenty second embodiment includes the vector according to any one of the twentieth and the twenty first embodiments, wherein the MAL1 gene cluster and/or a MAL2-8c gene cluster are maintained and expressed in a haploid, diploid, or polyploid of the strain of S. cerevisiae.

A twenty third embodiment includes the vector according to any one of the twentieth to the twenty second embodiments, wherein the vector is expressed in the strain of S. cerevisiae as a single copy or multiple copies. Consistent with these embodiments, the vector and/or plasmid may either be maintained at one copy per cell or as multiple copies per cell.

A twenty fourth embodiment includes a vector comprising a MAL2-8c gene cluster that comprises a sequence having 80 percent, 81 percent, 82 percent, 83 percent, 84 percent, 85 percent, 86 percent, 87 percent, 88 percent, 89 percent, 90 percent, 91 percent, 92 percent, 93 percent, 94 percent, 95 percent, 96 percent, 97 percent, 98 percent, 99 percent, and/or 100 percent homology or identity to SEQ ID NO: 3.

A twenty fifth embodiment includes the vector according to the twenty fourth embodiment, wherein the MAL2-8c gene cluster is maintained and expressed in a haploid, diploid, or polyploid of the strain of S. cerevisiae.

A twenty sixth embodiment includes the vector according to any one of the twenty fourth and the twenty fifth embodiments, wherein the vector is expressed in the strain of S. cerevisiae as a single copy or multiple copies.

A twenty seventh embodiment includes a method of producing a recombinant yeast strain, comprising: integrating the exogenous MAL1 gene cluster and/or the exogenous MAL2-8c gene according to any one of the first to the nineteenth embodiments into the genome of the strain of S. cerevisiae.

A twenty eighth embodiment includes the recombinant yeast strain according to any one of the first to the nineteenth embodiments, wherein the recombinant yeast strain is made using genetic engineering or wherein the recombinant yeast strain is genetically modified.

A twenty ninth embodiment includes any one of the first to the twenty eighth embodiments, wherein the recombinant yeast strain is capable of fermenting maltose as well as disaccharides and trisaccharides comprised of glucose while simultaneously improving the efficiency and speed of glucose fermentation.

The present invention relates to a recombinant yeast strain comprising a strain of S. cerevisiae and an exogenous MAL1 gene cluster comprising a MAL11 gene, a MAL12 gene and/or a MAL13 gene and an exogenous MAL2-8c gene cluster; the exogenous MAL1 gene cluster may comprise a sequence having at least 80 percent homology to SEQ ID NO: 2; the exogenous MAL2-8c gene cluster may comprise a sequence having at least 80 percent homology to SEQ ID NO: 3. The exogenous MAL1 gene cluster may be overexpressed. The MAL1 gene cluster may comprise a sequence having at least 85 percent homology to SEQ ID NO: 2 and the MAL2-8c gene cluster may comprise a sequence having at least 85 percent homology SEQ ID NO: 3. The MAL1 gene cluster may comprise a sequence having at least 90 percent homology to SEQ ID NO: 2 and the MAL2-8c gene cluster may comprise a sequence having at least 90 percent homology to SEQ ID NO: 3. The MAL1 gene cluster may comprise a sequence having at least 95 percent homology to SEQ ID NO: 2 and the MAL2-8c gene cluster may comprise a sequence having at least 95 percent homology to SEQ ID NO: 3. Maltose may comprise a weight of no more than 4.64 percent of the total volume after 24 hours of fermentation when present in a medium containing corn mash with 31.3 percent weight to weight solids treated with a 1 percent weight to weight solution of maltogenic alpha amylase. Maltose may comprise a weight of no more than 2.86 percent of the total volume after 36 hours of fermentation when present in a medium containing corn mash with 31.3 percent weight to weight solids treated with a 1 percent weight to weight solution of maltogenic alpha amylase. Maltose may comprise a weight of no more than 1.88 percent of the total volume after 48 hours of fermentation when present in a medium containing corn mash with 31.3 percent weight to weight solids treated with a 1 percent weight to weight solution of maltogenic alpha amylase. Maltose may comprise a weight of no more than 1.05 percent of the total volume after 72 hours of fermentation when present in a medium containing corn mash with 31.3 percent weight to weight solids treated with a 1 percent weight to weight solution of maltogenic alpha amylase. Ethanol may comprise a weight of at least 8.733 percent of the total volume after no more than 24 hours when present in fermentation of corn mash with 31.3 percent weight to weight solids treated with a 1 percent weight to weight solution of maltogenic alpha amylase. Ethanol may comprise a weight of at least 4.16 percent of the total volume after no more than 15 hours when present in fermentation of corn mash with 32.6 percent weight to weight solids treated with 0.07 percent weight to weight glucoamylase. Ethanol may comprise a weight of at least 10.35 percent of the total volume after no more than 26 hours when present in fermentation of corn mash with 32.6 percent weight to weight solids treated with 0.07 percent weight to weight glucoamylase. The MAL1 gene cluster may comprise a sequence having at least 85 percent homology to SEQ ID NO: 2 and the MAL2-8c gene cluster may comprise a sequence having at least 85 percent homology SEQ ID NO: 3. The MALL gene cluster may comprise a sequence having at least 90 percent homology to SEQ ID NO: 2 and the MAL2-8c gene cluster may comprise a sequence having at least 90 percent identity to SEQ ID NO: 3. The MAL1 gene cluster may comprise a sequence having at least 95 percent homology to SEQ ID NO: 2 and the MAL2-8c gene cluster may comprise a sequence having at least 95 percent homology to SEQ ID NO: 3. The MAL1 gene cluster may comprise a sequence having at least 98 percent homology to SEQ ID NO: 2 and the MAL2-8c gene may comprise a sequence having at least 95 percent identity to SEQ ID NO: 3.

The present invention relates to a recombinant yeast strain comprising a strain of S. cerevisiae and an exogenous MAL1 gene cluster and an exogenous MAL2-8c gene; the exogenous MAL1 gene cluster may comprise a sequence having at least 80 percent identity to SEQ ID NO: 2; the exogenous MAL2-8c gene may comprise a sequence having at least 80 percent identity to SEQ ID NO: 3. The exogenous MAL1 gene cluster may comprise a MAL11 gene, a MAL12 gene and/or a MAL13 gene. The exogenous MAL1 gene cluster may be overexpressed. The MAL2-8c gene may be inserted into the genome of the strain of S. cerevisiae within a region encoding the Dubious Open Reading Frame YEL028W.

### FIGURES

FIG. 1 is a schematic diagram comprising a schematic drawing illustrating DNA sequence analysis of Fermentis Ethanol Red® strain and alignment of sequencing reads with the MAL1 gene cluster of S288c.

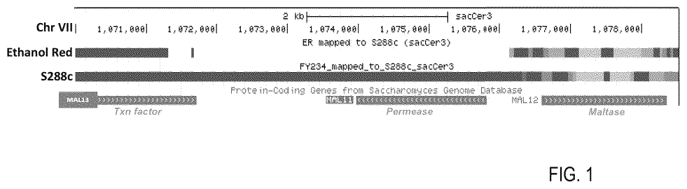

FIG. 2 is a schematic diagram comprising a schematic drawing illustrating strategy to replace the endogenous MAL1 gene cluster in Fermentis Ethanol Red® strain with MAL1 gene cluster from Cen.PK 113-7D strain.

FIG. 3 is a schematic diagram comprising a drawing illustrating construction of the MAL2-8c gene cassette using overlapping PCR fragments in the pDNLS2 vector targeting Neutral Landing Site 2 as the site of integration.

FIG. 4 is a schematic diagram comprising a drawing illustrating details of the genomic features and gene expression profiles around dubious ORF YEL028W, termed “Neutral Landing Site #2”, the site of MAL2-8c integration. YEL028W is a dubious Open reading frame whose transcript does not code for a functional protein. Gene expression values are shown. These values represent transcripts per million, a normalized method of measuring gene expression via RNA-Seq.

FIG. 5A is a schematic diagram comprising a graph illustrating the changes in DP4+ levels from wild type and the maltophilic strain under conditions of maltose and glucose co-fermentation when corn mash is treated with a 1 percent solution of maltogenic alpha amylase, SEB Star MA.

FIG. 5B is a schematic diagram comprising a graph illustrating the changes in DP3 levels from wild type and the maltophilic strain under conditions of maltose and glucose co-fermentation when corn mash is treated with a 1 percent solution of maltogenic alpha amylase, SEBStar MA.

FIG. 5C is a schematic diagram comprising a graph illustrating the changes in maltose levels from wild type and the maltophilic strain under conditions of maltose and glucose co-fermentation when corn mash is treated with a 1 percent solution of maltogenic alpha amylase, SEBStar MA.

FIG. 5D is a schematic diagram comprising a graph illustrating the changes in glucose levels from wild type and the maltophilic strain under conditions of maltose and glucose co-fermentation when corn mash is treated with a 1 percent solution of maltogenic alpha amylase, SEBStar MA.

FIG. 5E is a schematic diagram comprising a graph illustrating the changes in ethanol levels from wild type and the maltophilic strain under conditions of maltose and glucose co-fermentation when corn mash is treated with a 1 percent solution of maltogenic alpha amylase, SEBStar MA.

FIG. 6A is a schematic diagram comprising a graph illustrating the changes in DP4+levels from wild type and the maltophilic strain under conditions of maltose and glucose co-fermentation when corn mash is treated with SEBStar MA (1 percent) and a low level (0.015 percent w/w) of CTE Global Glucoamylase.

FIG. 6B is a schematic diagram comprising a graph illustrating the changes in DP3 levels from wild type and the maltophilic strain under conditions of maltose and glucose co-fermentation when corn mash is treated with SEBStar MA (1 percent) and a low level (0.015 percent w/w) of CTE Global Glucoamylase.

FIG. 6C is a schematic diagram comprising a graph illustrating the changes in maltose levels from wild type and the maltophilic strain under conditions of maltose and glucose co-fermentation when corn mash is treated with SEBStar MA (1 percent) and a low level (0.015 percent w/w) of CTE Global Glucoamylase.

FIG. 6D is a schematic diagram comprising a graph illustrating the changes in glucose levels from wild type and the maltophilic strain under conditions of maltose and glucose co-fermentation when corn mash is treated with SEBStar MA (1 percent) and a low level (0.015 percent w/w) of CTE Global Glucoamylase.

FIG. 6E is a schematic diagram comprising a graph illustrating the changes in ethanol levels from wild type and the maltophilic strain under conditions of maltose and glucose co-fermentation when corn mash is treated with SEBStar MA (1 percent) and a low level (0.015 percent w/w) of CTE Global Glucoamylase.

FIG. 7A is a schematic diagram comprising a graph illustrating the changes in DP4+ levels from wild type and the maltophilic strain under conditions of maltose and glucose co-fermentation when corn mash is treated with Spirizyme Achieve Glucoamylase.

FIG. 7B is a schematic diagram comprising a graph illustrating the changes in DP3 levels from wild type and the maltophilic strain under conditions of maltose and glucose co-fermentation when corn mash is treated with Spirizyme Achieve Glucoamylase.

FIG. 7C is a schematic diagram comprising a graph illustrating the changes in maltose levels from wild type and the maltophilic strain under conditions of maltose and glucose co-fermentation when corn mash is treated with Spirizyme Achieve Glucoamylase.

FIG. 7D is a schematic diagram comprising a graph illustrating the changes in glucose levels from wild type and the maltophilic strain under conditions of maltose and glucose co-fermentation when corn mash is treated with Spirizyme Achieve Glucoamylase.

FIG. 7E is a schematic diagram comprising a graph illustrating the changes in ethanol levels from wild type and the maltophilic strain under conditions of maltose and glucose co-fermentation when corn mash is treated with Spirizyme Achieve Glucoamylase.

FIG. 8A is a schematic diagram comprising a graph illustrating the changes in DP4+ levels from wild type and the maltophilic strain under conditions of maltose and glucose co-fermentation when corn mash is treated with Spirizyme Achieve Glucoamylase at either 0.06 percent or 0.03 percent (w/w).

FIG. 8B is a schematic diagram comprising a graph illustrating the changes in DP3 levels from wild type and the maltophilic strain under conditions of maltose and glucose co-fermentation when corn mash is treated with Spirizyme Achieve Glucoamylase at either 0.06 percent or 0.03 percent (w/w).

FIG. 8C is a schematic diagram comprising a graph illustrating the changes in maltose levels from wild type and the maltophilic strain under conditions of maltose and glucose co-fermentation when corn mash is treated with Spirizyme Achieve Glucoamylase at either 0.06 percent or 0.03 percent (w/w).

FIG. 8D is a schematic diagram comprising a graph illustrating the changes in glucose levels from wild type and the maltophilic strain under conditions of maltose and glucose co-fermentation when corn mash is treated with Spirizyme Achieve Glucoamylase at either 0.06 percent or 0.03 percent (w/w).

FIG. 8E is a schematic diagram comprising a graph illustrating the changes in ethanol levels from wild type and the maltophilic strain under conditions of maltose and glucose co-fermentation when corn mash is treated with Spirizyme Achieve Glucoamylase at either 0.06 percent or 0.03 percent (w/w).

### DETAILED DESCRIPTION

For the purposes of promoting an understanding of the principles of the novel technology, reference will now be made to the preferred embodiments thereof, and special language will be used to describe the same. It will nevertheless be understood that no limitation of the scope of the novel technology is thereby intended, such alterations, modifications, and further applications of the principles of the novel technology being contemplated as would normally occur to one skilled in the art to which the novel technology relates.

As used herein, unless specified otherwise, the term ‘about’ means plus or minus 20 percent, for example, about 1.0 encompasses the range 0.8 to 1.2.

Unless specifically referred to otherwise, genes are referred to using the nomenclature suggested by Demerec et al., A proposal for a uniform nomenclature in bacterial genetics. J. GEN. MICROBIOL (1968) 50, 1-14.

A “vector” is any nucleic acid molecule for the cloning of and/or transfer of a nucleic acid into a cell. A vector may be a replicon to which another nucleotide sequence may be attached to allow for replication of the attached nucleotide sequence.

A “recombinant” vector refers to a viral or non-viral vector that comprises one or more exogenous nucleotide sequences (i.e., trans genes), e.g., two, three, four, five or more exogenous nucleotide sequences. An “expression” vector refers to a viral or non-viral vector that is designed to express a product encoded by an exogenous nucleotide sequence inserted into the vector.

The term “exogenous” with respect to a polynucleotide means a polynucleotide that is not native to the cell in which it is located or, alternatively, a polynucleotide which is normally found in the cell but is in a different location or is expressing different copy number than normal (e.g., in a vector or in a different location in the genome).

The term “recombinant organism” refers to any organism including, but is not limited to, a strain or a part of yeast whose genetic material has been altered using genetic engineering techniques. In any one of the embodiments disclosed herein, the polynucleotide can be inserted into a cell of an organism including, but is not limited to, a strain or a part of yeast by genetic engineering (e.g., insertion of an expression vector).

The term “express” or “expression” of a polynucleotide coding sequence means that the sequence is transcribed, and optionally, translated. Typically, according to the present invention, expression of a coding sequence of the invention will result in production of the polypeptide of the invention. The entire expressed polypeptide or fragment can also function in intact cells without purification.

As used herein, the terms “protein” and “polypeptide” can be interchangeably used and can encompass both peptides and proteins, unless specifically indicated otherwise.

For those skilled in the art, protein sequence similarity is calculated by alignment of two protein sequences. Commonly used pairwise alignment tools include COBALT (Papadopoulos and Agarwala, 2007), EMBOSS Needle (Needleman and Wunsch, 1970) and EMBOSS Stretcher (Myers and Miller, 1988). The percentage of identity represents the total fraction of amino acids that are identical along the length of each protein. Similarity is calculated based on the percentage of amino acids with similar character over the reported aligned region. Amino acids are considered similar if they share common chemical properties that impart similar qualities to the structure and activity of the entire protein.

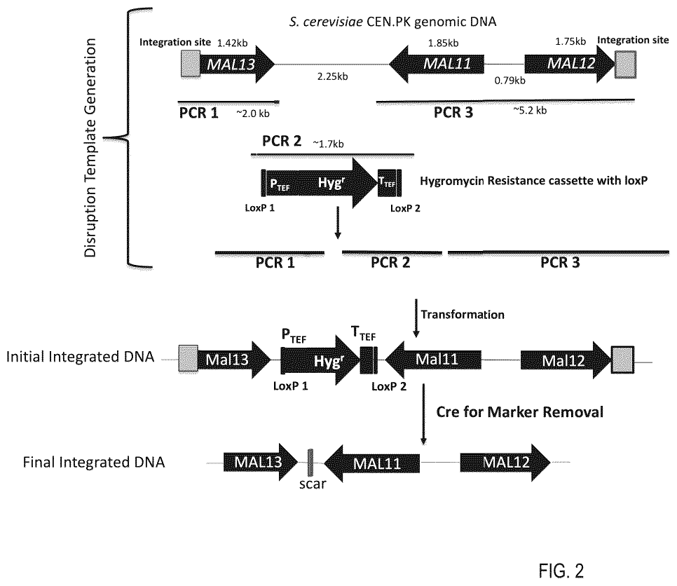

The endogenous MAL1 locus was modified using direct transformation with three overlapping PCR fragments (FIG. 2). PCR product numbers one and three were generated from genomic DNA template of strain CEN.PK 113-7D. PCR product number two was generated using a yeast expression vector pKC2 as template to amplify a hygromycin resistance gene flanked by LoxP sites allowing for removal by CRE Recombinase. After confirming integration of PCR products, the hygromycin resistance gene was removed, leaving a strain with a small DNA scar and no antibiotic resistance or other foreign genes (FIG. 2). Detailed sequence information is shown in the Sequence Listing section below.

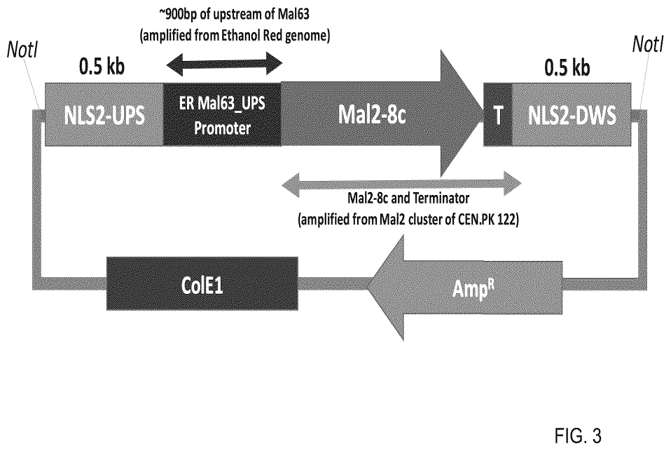

The MAL2-8c gene with its native terminator from S. cerevisiae CEN.PK 122 and promoter region from S. cerevisiae strain Fermentis Ethanol Red® strain (for details see Sequence list, MAL2-8c construct) were PCR amplified from the genomic DNA of the respective strains using Q5 PCR reaction mixture (New England Biolabs). The overlapping PCR fragments were gel purified and then cloned into PmeI linearized target vector backbone of pDNLS2 (FIG. 3) using HiFi DNA assembly kit as recommended in the manufacturer's protocol (New England Biolabs). The correct vector assembly with desired genetic components was verified by PCR and sequencing. The DNA of verified MAL2-8c gene cassette was digested with NotI restriction enzyme and gel purified as linear DNA fragments for integration into the designated Neutral Landing Site 2 of selected S. cerevisiae strains using CRISPR technology. The linear DNA fragment of MAL2-8c cassette and plasmid DNA expressing both the nuclease and NLS2-targeting SgRNA were transformed into S. cerevisiae according to a previously published protocol (Gietz et al., Yeast transformation by the LiAc/SS Carrier DNA/PEG method, METHODS MOL BIOL 2006, 313:107-120). The transformed cells were plated on selective YPD media plates supplemented with 50 μg/ml of G418 antibiotic. Plates were incubated at 30° C. for 2-3 days, until colonies became visible. Upon appearance of visible colonies on YPD plates, integration of MAL2-8c gene cassette at the NLS2 site was confirmed via direct colony PCR prior to long term storage in 15 percent glycerol at −80° C. The resulting strain is known to us as ER-19-11-4.

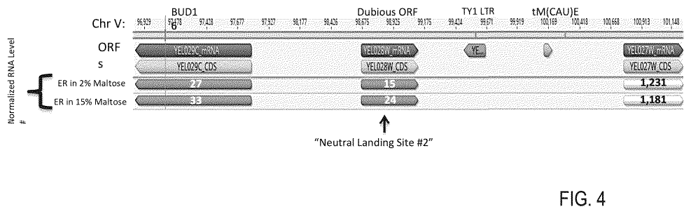

Neutral Landing Site 2 (NLS2) was selected as the site of MAL2-8c integration for several regions. First, to avoid disrupting any important genetic elements; a spot-on chromosome V overlapping the dubious open reading frame YEL028W but sufficiently distant from other annotated genes was chosen. Genome-wide RNA expressions were measured in Fermentis Ethanol Red® fermenting either maltose or glucose at both high (15 percent) and low (2 percent) concentrations. Under all conditions tested the genes neighboring NLS2 are expressed at moderate levels indicating that this is a region amenable to Pol II transcription under a wide variety of conditions (FIG. 4). Together the analyses disclosed herein indicate the region overlapping YEL028W provides a suitable and stable platform where superior genetic traits can be engineered in Ethanol Red® and their derivative strains.

### Experimental

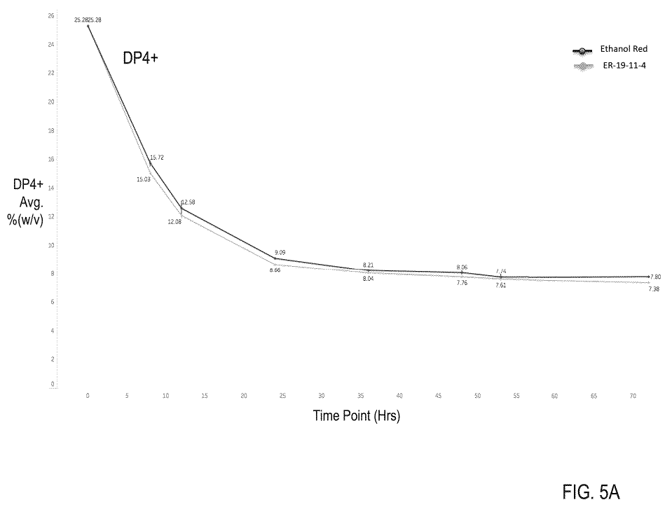

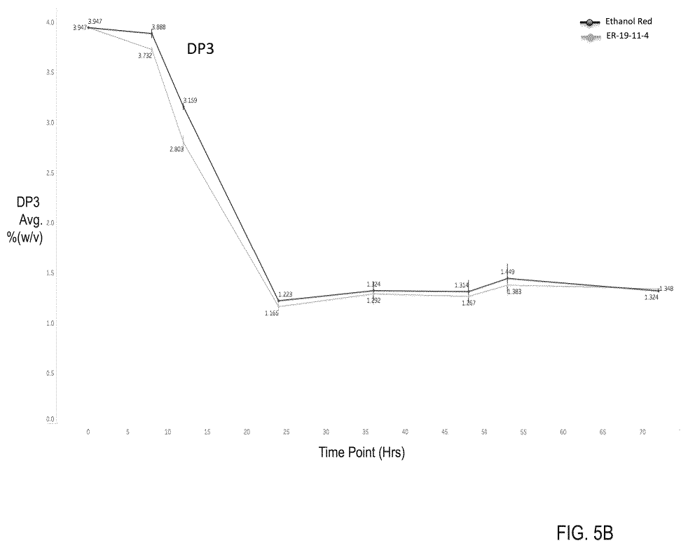

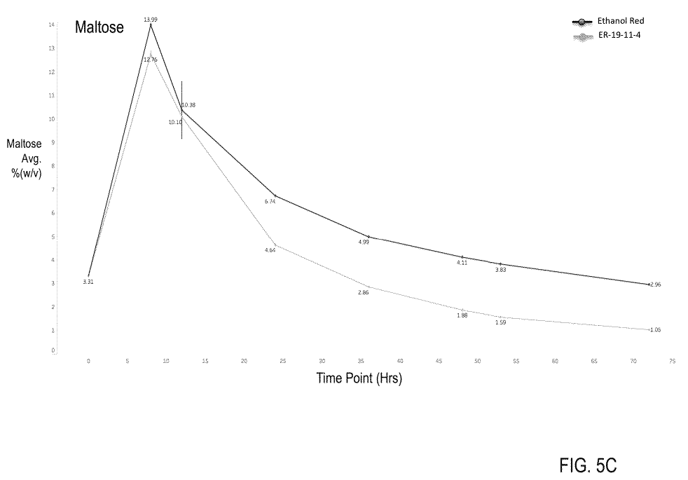

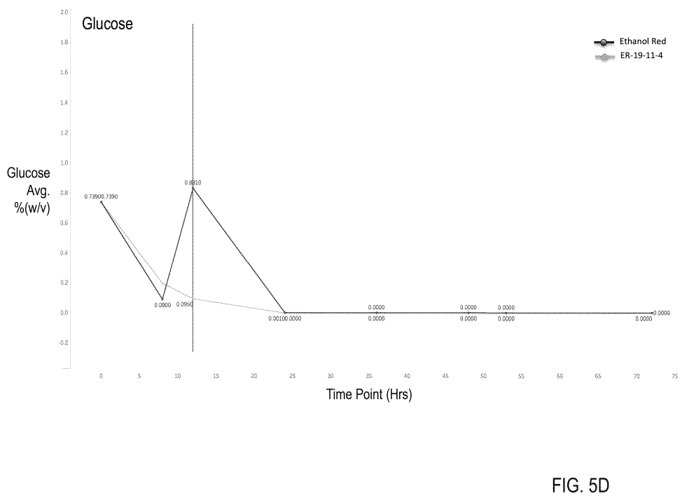

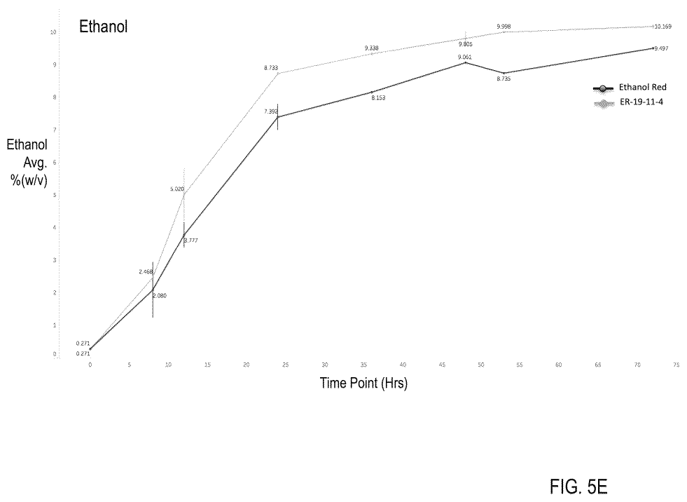

To test the fermentation ability of ER-19-11-4, corn mash containing 31.3 percent solids was treated with a 1 percent solution of maltogenic alpha amylase SEBStar MA (Specialty Enzymes). Maltogenic strain ER-19-11-4 produced more ethanol than Fermentis Ethanol Red® at all time points, including fermentation finish (FIG. 5F). Higher ethanol production by ER-19-11-4 is due primarily to increased maltose consumption (FIG. 5C). ER-19-11-4finished fermentation with only 1.05 percent (w/v) maltose remaining while the unmodified Ethanol Red® strain left 2.45 percent (w/v) maltose at the end of fermentation. Both strains finished with equivalent levels of DP3 sugars but the maltophilic yeast ER-19-11-4 consumed DP3 quicker than Ethanol Red®, up until 24 hours when both fermentations reached a steady state (FIG. 5B).

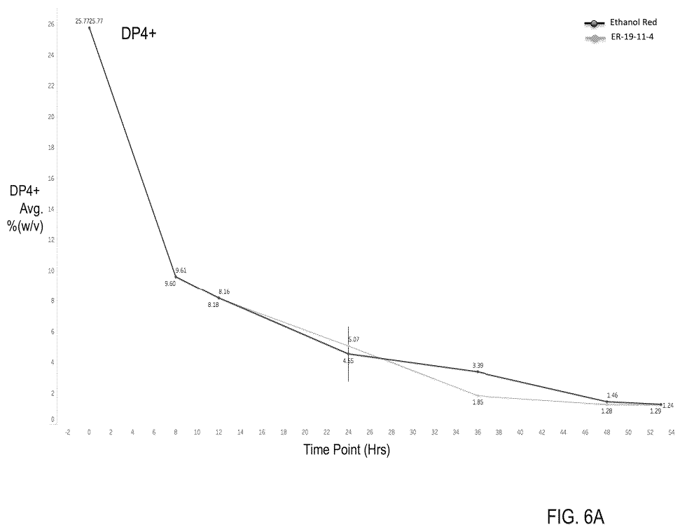

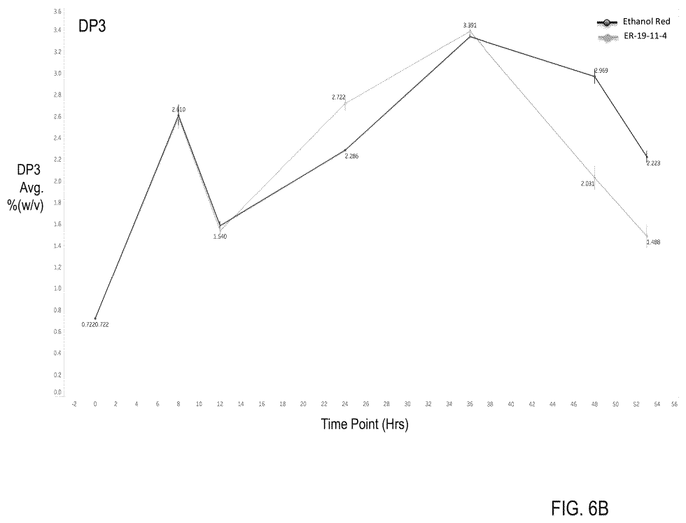

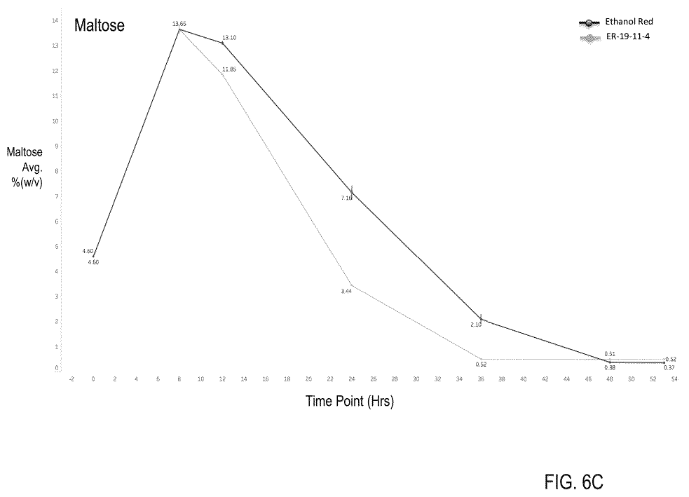

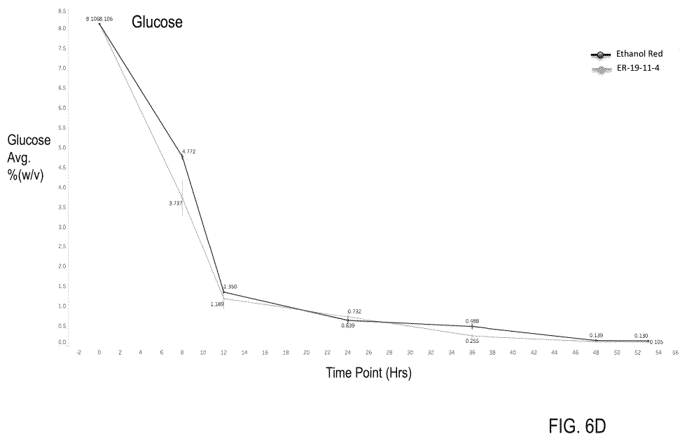

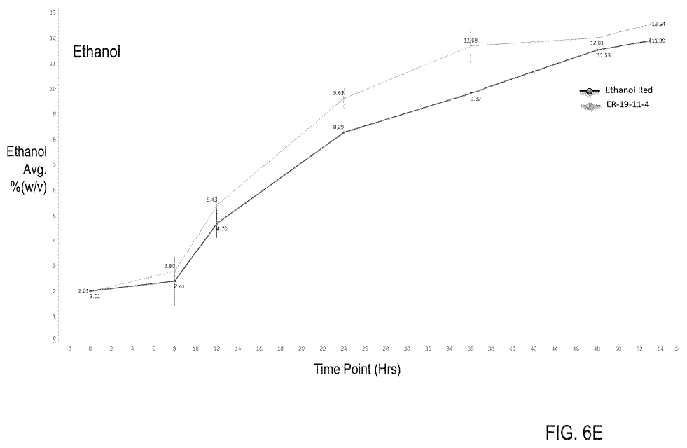

As a second test, corn mash with 31.3 percent solids was treated with 1 percent SEBStar MA and a low level (0.015 percent w/w) of CTE Global Glucoamylase. The combined enzyme treatment resulted in more DP4+ breakdown, higher glucose levels, higher final ethanol levels while still producing high maltose syrups (FIGS. 6A-E). Under these conditions, maltophilic yeast ER-19-11-4 consumed maltose faster and produced more ethanol than an isogenic wild type strain (FIG. 6E). ER-19-11-4 also showed slightly improved glucose consumption (FIG. 6F). Combining maltogenic alpha amylase and glucoamylase resulted in more DP3 fluctuation than maltogenic alpha alone; however, after 36 hours the ER-19-11-4 strain consumed more DP3 sugars than Ethanol Red® and final DP3 values at 54 hours were significantly lower in the ER-19-11-4 fermentations (1.5 percent) compared to Ethanol Red® fermentations (2.2 percent) (FIG. 6B).

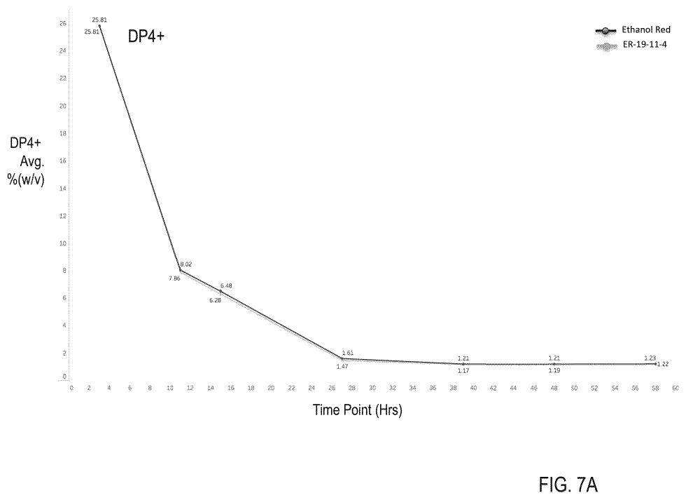

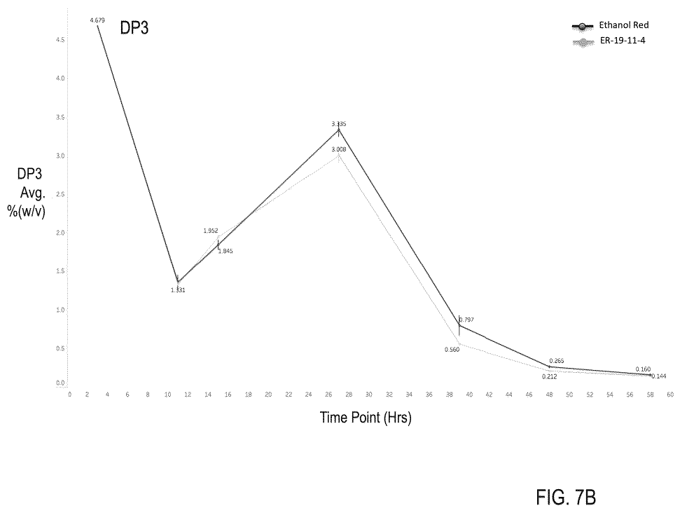

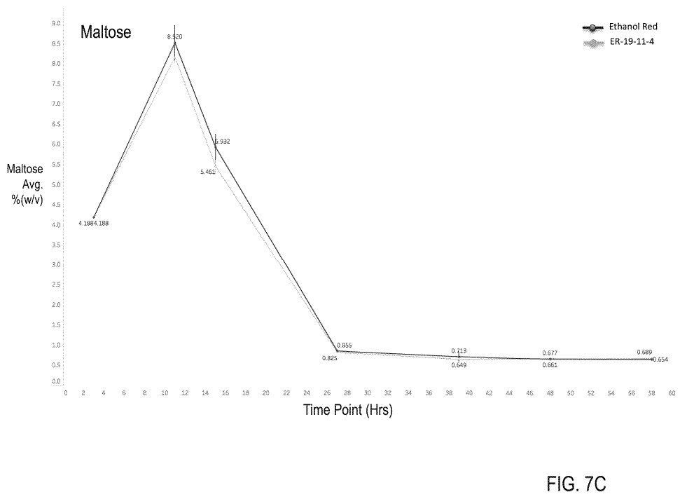

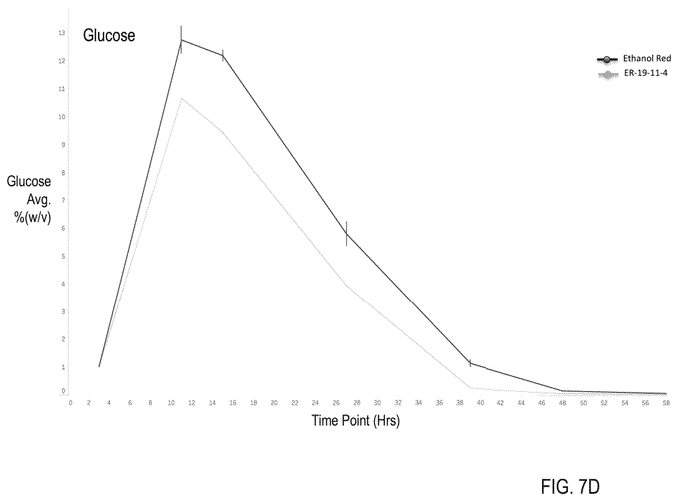

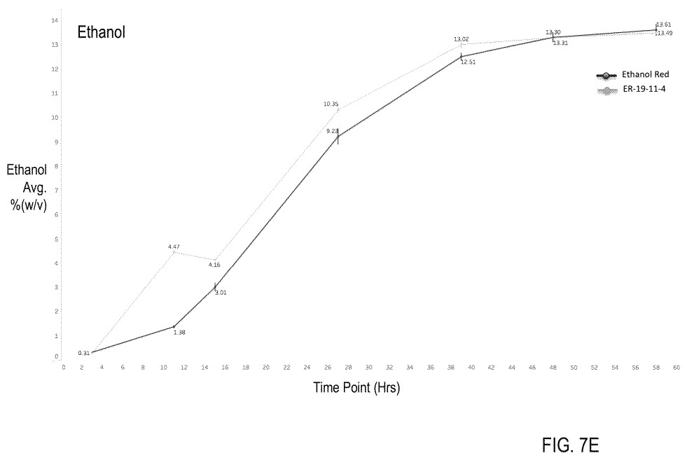

As a third test, corn mash with 32.6 percent solids was treated with (0.07 percent w/w) of Spirizyme Achieve Glucoamylase (Novozymes). This higher glucoamylase enzyme treatment resulted in even higher glucose levels along with higher final ethanol levels (FIGS. 7C-E). Under these conditions, maltophilic yeast ER-19-11-4 consumed maltose slightly faster and again produced more ethanol than an isogenic wild type strain (FIG. 7E). ER-19-11-4 also showed significant improvement in the rate of glucose consumption (FIG. 7F).

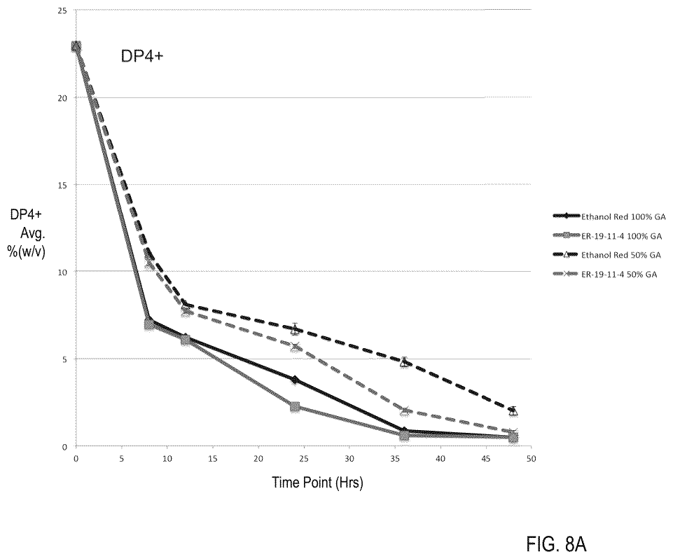

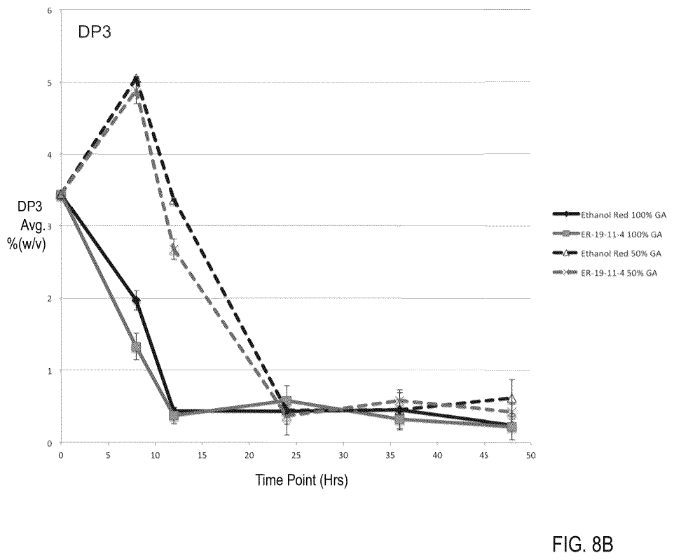

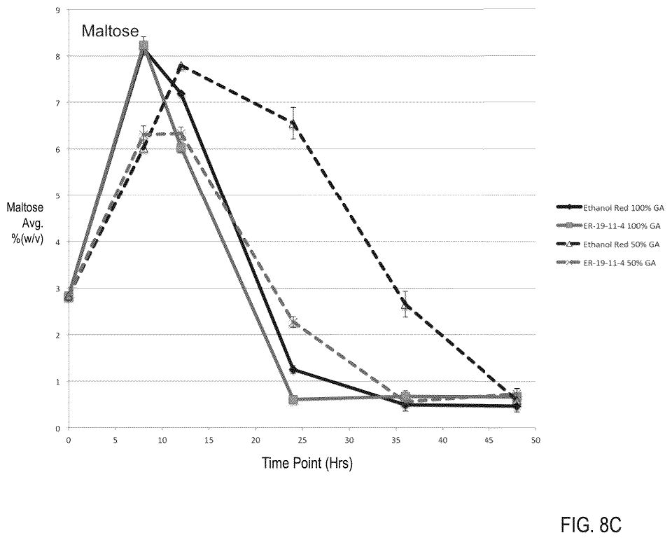

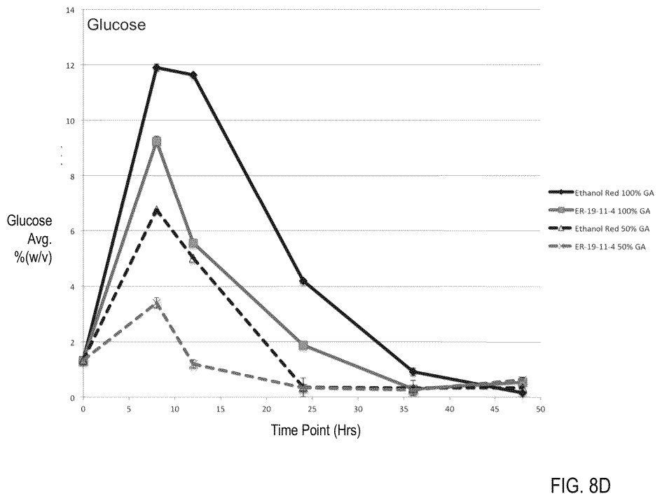

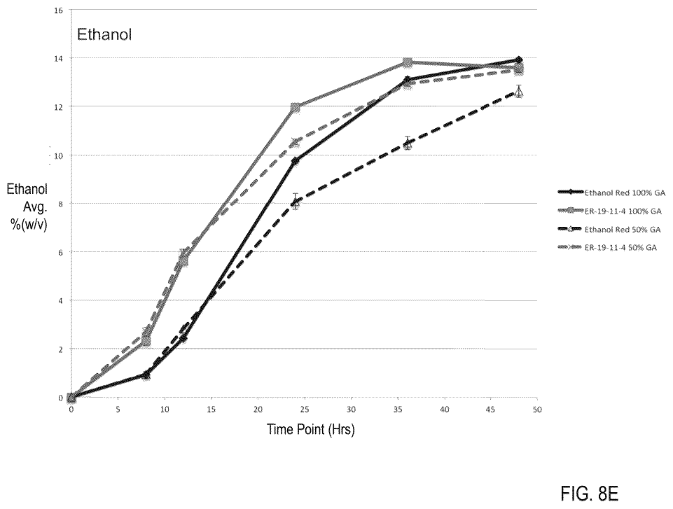

As a final test, corn mash with 32 percent solids was treated with either a full dose (0.06 percent w/w) of Spirizyme Achieve Glucoamylase (Novozymes) or a half dose (0.03 percent w/w). Again, at the GA dose, ER-19-11-4 consumed DP4+, DP3, maltose and glucose faster and reached maximal ethanol levels at least 10 hours earlier than wild type at an increased rate (FIG. 8A-E). Reducing the amount of glucoamylase represents a chance for significant cost savings for fuel ethanol plants. In fermentations with a half dose of GA, excess DP4+ sugars remained at fermentation finish for the wild type strain, resulting in lower final ethanol concentration. Importantly, at 50 percent GA, the ER-19-11-4 strain allows for full DP4+ consumption and produces final ethanol concentrations equivalent to the wild type strain at 100 percent GA (FIG. 8A, E). The rate of ethanol production is also quicker for the ER-19-11-4 at 50 percent. This opportunity for enzyme cost savings was unexpected prior to experimentation and we suspect that increased rate of glucose and maltose consumption by the maltogenic strain allows the glucoamylase to work more efficiently. Overall, ER-19-11-4 shows improved maltose and glucose consumption and in turn increased ethanol yields over a wide range of fermentation conditions. Furthermore, this strain requires significantly less glucoamylase than the amount used with other leading industrial strains.

The invention claimed is:

1. A recombinant yeast strain comprising:
a strain of S. cerevisiae;
an exogenous MAL1 gene cluster comprising a MAL11 gene, a MAL12 gene and/or a MAL13 gene;
an exogenous MAL2-8c gene cluster;
wherein the exogenous MAL1 gene cluster comprises a sequence having at least 80 percent homology to SEQ ID NO: 2; and
wherein the exogenous MAL2-8c gene cluster comprises a sequence having at least 80 percent homology to SEQ ID NO: 3.

2. The strain of claim 1 wherein the exogenous MAL1 gene cluster is overexpressed.

3. The strain of claim 1 wherein the MAL1 gene cluster comprises a sequence having at least 85 percent homology to SEQ ID NO: 2 and the MAL2-8c gene cluster comprises a sequence having at least 85 percent homology SEQ ID NO: 3.

4. The strain of claim 1 wherein the MAL1 gene cluster comprises a sequence having at least 90 percent homology to SEQ ID NO: 2 and the MAL2-8c gene cluster comprises a sequence having at least 90 percent homology to SEQ ID NO: 3.

5. The strain of claim 1 wherein the MAL1 gene cluster comprises a sequence having at least 95 percent homology to SEQ ID NO: 2 and the MAL2-8c gene cluster comprises a sequence having at least 95 percent homology to SEQ ID NO: 3.

6. The strain of claim 1 wherein maltose comprises a weight of no more than 4.64 percent of the total volume after 24 hours of fermentation when present in a medium containing corn mash with 31.3 percent weight to weight solids treated with a 1 percent weight to weight solution of maltogenic alpha amylase.

7. The strain of claim 1 wherein maltose comprises a weight of no more than 2.86 percent of the total volume after 36 hours of fermentation when present in a medium containing corn mash with 31.3 percent weight to weight solids treated with a 1 percent weight to weight solution of maltogenic alpha amylase.

1. train of claim 1 wherein maltose comprises a weight of no more than 1.88 percent of the total volume after 48 hours of fermentation when present in a medium containing corn mash with 31.3 percent weight to weight solids treated with a 1 percent weight to weight solution of maltogenic alpha amylase.

9. The strain of claim 1 wherein maltose comprises a weight of no more than 1.05 percent of the total volume after 72 hours of fermentation when present in a medium containing corn mash with 31.3 percent weight to weight solids treated with a 1 percent weight to weight solution of maltogenic alpha amylase.

10. The strain of claim 1 wherein ethanol comprises a weight of at least 8.733 percent of the total volume after no more than 24 hours when present in fermentation of corn mash with 31.3 percent weight to weight solids treated with a 1 percent weight to weight solution of maltogenic alpha amylase.

11. The strain of claim 1 wherein ethanol comprises a weight of at least 4.16 percent of the total volume after no more than 15 hours when present in fermentation of corn mash with 32.6 percent weight to weight solids treated with 0.07 percent weight to weight glucoamylase.

12. The strain of claim 1 wherein ethanol comprises a weight of at least 10.35 percent of the total volume after no more than 26 hours when present in fermentation of corn mash with 32.6 percent weight to weight solids treated with 0.07 percent weight to weight glucoamylase.

13. The strain of claim 1 wherein the MAL1 gene cluster comprises a sequence having at least 85 percent homology to SEQ ID NO: 2 and the MAL2-8c gene cluster comprises a sequence having at least 85 percent homology SEQ ID NO: 3.

14. The strain of claim 1 wherein the MAL1 gene cluster comprises a sequence having at least 90 percent homology to SEQ ID NO: 2 and the MAL2-8c gene cluster comprises a sequence having at least 90 percent identity to SEQ ID NO: 3.

15. The strain of claim 1 wherein the MAL1 gene cluster comprises a sequence having at least 95 percent homology to SEQ ID NO: 2 and the MAL2-8c gene cluster comprises a sequence having at least 95 percent homology to SEQ ID NO: 3.

16. The strain of claim 1 wherein the MAL1 gene cluster comprises a sequence having at least 98 percent homology to SEQ ID NO: 2 and the MAL2-8c gene comprises a sequence having at least 95 percent identity to SEQ ID NO: 3.

17. A recombinant yeast strain comprising:
a strain of S. cerevisiae;
an exogenous MAL1 gene cluster;
an exogenous MAL2-8c gene;
wherein the exogenous MAL1 gene cluster comprises a sequence having at least 80 percent identity to SEQ ID NO: 2; and
wherein the exogenous MAL2-8c gene comprises a sequence having at least 80 percent identity to SEQ ID NO: 3.

18. The strain of claim 17 wherein the exogenous MAL1 gene cluster comprises a MAL11 gene, a MAL12 gene and/or a MAL13 gene.

19. The strain of claim 17 wherein the exogenous MAL1 gene cluster is overexpressed.

20. The strain of claim 17 wherein the MAL2-8c gene is inserted into the genome of the strain of S. cerevisiae within a region encoding the Dubious Open Reading Frame YEL028W.


SEQUENCE LISTING (6 sequences)

SEQ ID NO: 1 - DNA, 8738 bp, Saccharomyces cerevisiae
  source 1..8738: mol_type = unassigned DNA
caccccagccatcgtcattagagtctttgaaacttgctgggtaaatttgatagaacgtggcctctttccaccactttggttctgtctctggatgtgcagaagaaatagtcatcgatagtaaatattacgttgaaaagctttgtttgtatcttgtttgatctgtgcttgctcgattaatttgagacagcttttttatttcaaaacaggcgtctaaccaaacgtctagcaactcaatatcattgcccttaagtactttttatttcaaaaggagatcctttctcattctggggtaaactatggtatgacgaaaaccatgaaaaataaggaacataatttatccgagtatttcaacgatcccaagtactgacataaactttagtagccaatttatagcgtggggtgcctacttcgtcacatttgatatcgtacagcgaaaaaacattagtaactttatttccttatttcagggccacttttctcgagaataacgctgcgtgctgagcggttgttcacaccgcggagttggaaactttattctccgaaatattctccccactaaaatatccttacgtattgtgaaacttagttttctttttgtattagggtgtaatttcttattttccctgtatttcaccgcatgcaaattcttacgatatttactccggtaaacgcagttaagagctattgtccggtccgactgaatgaatattcggttagaaacgcatatttgtggggaaataacaacctcaaagatatagacggagcagtaccgtaaggtttacagaatggcatgaccacccacaataaagcaaggacctcgagacacatgcctttcaaaatagaaataaaggttttcgaacatcatttttcgcttgttgtatagtagtctttacagtaacagtgcatctgagtacaggaacgattgtcttgataatatgtgaaaagtgcacacaaaattagagggtgtcctttacaagtattcttagaaacacattcaagagcacaaaagtcgatgctttaagggtcaaggtggtggaaaacttgactggaattcttgacgaaaaaacaagaaaaacgtgattcgagcaatcataaacatacagccccgttccaaccggatcttgaggtttcccattttagatggaaataagcagagcaaaataaaaatcttgaacaagtaatagtggtgactgcaggttacgttggcatataaagtccgggtgacctgggtttcctgcaccaccagcccccatatgctagcacaatgggttttctttatccccggtcataattactcattttgctatattcttcataacttaagtacgcagatagagaaaattaataatctcgatatatattaaagtaaatgaaaagtagaaaatttagccagaactcttttttgcttcgagtatgactttaactaagcaaacatgcgccaagcaggcatgcgactgctgtcgtattcgtcgagtgaaatgcgatggtaaaaggccgtgtagcagttgcctacagaatagtttggattgcacttatctgcaaccgtcgagaaaaagaggtccgaagtccattaggttgaggagcttgaaaagaatagcagaagtgcagagggaaagcggtcctaacaccattgcaactgctcctgtaatatataagagggttcccaaaaagctaatcgatcagtgcttgcggctctatcacgataatttatacgtaatctggccccttctttcgtacgatgaccttcacaaacttctggaggaaaaatacaatgacaattacgtatattggtttctgaccgctttatcagcggccaccctcagtgatttacaaactgaaataaaatctgaagaggaagtcactttcacgggaaaacggttatctaatctttgcatctcatcgtgtcagcaattcgacgatttggataacagcaatatattcaatattatgacgtactactgtttgcatcgtagctttgcacaaatatcgaacgcaagaacttcttacagactctgttgtgaagcggtcggtctgattacggtagcagggttacatcgggaagaaacttacggatcccttacatttgaagaacagcaacttagacggaaactttattacttgcttctcatgacggagagatactatgccatatatcttcattgtgcgacgagcctggatgccacaatagcaccaccgcaacttgaacttgtaactgatcctcagctttctatggacagtttccttgaaatgattagggtatttactgtaccaggaaaatgtttcttcgatgctttagccgctgactctacagatgcttcttgcactgaagagtcattgaaaaagatatggaaagagctccatacagcatcattagaaatagagccgtggtcttacggttacgttgacattgcattttccaggcactggattagagtcctcgcttggaagctagtcttgcggacaggaaatatcaacttcctatccgcctctaacagtgcacatgtaccacttgaaattgcaagggatatgcttgacgacgtgtttctaacaccaaataatctttatggagttcatggccctgggataccaacaaaggcaatagaagtagccaatgcactagtggatgtcatgaatcagtatgatcaagatactgaatcagaggcttggaaagttttgtgcgaaatttccaaatttgtcttctctttaaaacaatacgatggaaaactggttgaaaattttgtgactaaatgtcagagcgctcttattactcttccaatctctaaacctttgaaaaaaaatgaagatttgcataaaatatgactcactttaatttcttgagtgaacatttttcatccatttcttcatgtaaactccaaaaaagaaagcttctgtcggttttaagataaaagtactcctcgtgtataggaatatttttatctttgttagctctgtagaaaaagatacagataaagctcctgtaatatttgttgcagatttttggtccatgaattattttatcacgatcgaaaagaagttatgttcgttatatccagtggaaaaagcattgttatatgatggagtccggtgcctgtctctgcataaaaaaataacattttaatacatgggaggtgttatattgtacagagaggagacaatgatatggctttgttggtgttgtatgactaacatagggcgtttttatgattcatgaaatttatttaatacatgtttacgattttaactattgtgaatacattgctattgtatatatgtaatcatatcagcaattctagcattttaacatgtgacttgagctggatgattaaaatatgttaatttttttagaattattatctagtacaacaactaccagaatagttgaactgaataatatcaaatgaaaaggactcctctagctgaattttggaatgtttgccaaataaaaaaaagactttataacaaaaggttaattaaatgtatttagtaaaaaaaaaaaagtttgtcatatttatctattgaaatgaagtatcatttgttcacaacagatgaggtgcttcgcccttcatctaccacagaagtttccaaatcttccttcggatctttaacattaatttctgcagctgctgctttggcagctgcaaaagggtcgactttagtcgacttgaactttcttgctggaacaccaagtctaaacaattcatttatctcaataaaagtcctgccagcggtttctggtaaatcgacaacagcccaagctaaagtggccagacaaaatcctccccagaaaaagcctgatttagcaccccaattccatttctctgagttcaattggtacatgatcaaaactgtaactacaacttggatcacattgtaagcattacgagccaaaataattgttttggttcttagccttgaagacggtatttcagacactaagcaaaaaacaacaggtgcaataccgaggttgtaaaagaacgcgacaaccattagaagagcaccactacccattttagcgccatgagtgtctgaacatcctaaaccaccgataatgaagaacataatagcctgaaaagccagcccaaaagcataaaggtcaaatctgccacaatattttgaagcccaccaggatataaacgttgcagcaataccaagacaatattggataatactgaaagtaaaagccgtatcagtgctaacaccagctttttcataaaagtaagttgaataaccaattaatgatgcaccacaggagcattgaccgatccaacataaacaagctattctcgttcttctcctgttaataccatctttcacacaatcccagtaagttccttcatcagacattttctgctccttttctatagtagttttgattttatcgagttccatactcactagtaattctttctcgggtcctttaccacttaatgttctttcaagtgatctcctcgcttgatcaatccttccttttttaaccagccaccatggagactctggtgcaaaaaaaatacctaccgccaaaggaaggggccagatccactgcaaagcaaaaggtagcttatatcctagttctgagttggcatatttgttctgggaatttttcataataccagcagcgaaaagttgaccgaacgcccaacataaattagaataagtcgtcaaatagtatcttagggccaaaggacaaatttcagaagcataagaaacggtcaaacattggaaacaaccccatggcataccacacaatgcctgtcccacggcaatcatacccaaactcttgcaaaaatacagaatgaaaatgaaagccgctaaaaagaacaacgccatgatcagagtgtaacggttgcccatgtaatctacagaaggcccagtcatttgcaaaccgacaatctcacctgccatgtagcatagacatagaccgatttgccaggaaactgaaatttcataatctcctgtattgctattcaaagaaccatattttttttgaaaaacaggcagggcatagaaagctcctagaatggctgtgtcataaccctcttgaatcaatgttgtggaaactaatagtgaccaagcagcagcttttggatatgtcttcaaagctgtcatgagtggcattcccctctcactttcatctgcctctttggcgtcctgcatagcttcatcgagaaggtcggggacttcttcattattatcgtttggtattagtgaacctggaccgtactcaagatgggaaagatcaaaatcacttttcttaccttgctcctccatctctatcgagttgaattcggtagcgttcacgccattctcgatctcatctaagtgtgagtcgttcctgtctttttttctgtttattaatgaggataatcccttcatagttaattaatagtcttggatgtaattcttattgttatactgaatatgctaaaaccactcacaacaagtatggagtatattgtgcctctttatatcctgagtacttatgcaatatgcgctcactcaggatgaaatgtacacagccgaaagtatattgaaagctgcctctgcggaaacttctatctaatgttgtctccagatgtagactatgaggcctgaagaagtctttaagcacctgttggagagtataaggagactgctacaacaacgtcttccccacaaaaaattatgtggaggccgctatgatacctgcacaaacgttaagttacacatgaaaaagagactgacataactttgatctctgaaaatatgttttccccgagtagcttcactgcttggataccaatacgaatagaccttggctatagtaagttgcctctgtaccgtagagattcttgcaacctcgcttaaactctcgcttttatcaaatttcgctaaacacggggtttaagttaaagtttacaggatttatccggaaattttcgcggaccccacacaattaagaattggctcgaagagtgataacgcatacttttcttttctttttttagttcctagcgtacctaacgtaggtaacatgatttggatcgtgggatgatacaaacaacgtaagatgaatagttccttcctcaattcttcttgcagcatcattttcttgaggcgctctgggcaaggtataaaaagttccattaatacgtctctaaaaaattaaatcatccatctcttaagcagtttttttgataatctcaaatgtacatcagtcaagcgtaactaaattacataaatgactatttctgatcatccagaaacagaaccaaagtggtggaaagaggccacaatctatcaaatttacccagcaagttttaaagactccaataacgatggctggggtgatttaaaaggtatcacttccaagttgcagtatattaaagatcttggcgttgatgctatttgggtttgtccgttttatgactctcctcaacaagatatggggtatgatatatccaactacgaaaaggtctggcccacatacggtaccaatgaggactgttttgagctaattgacaagactcataagctgggtatgaaattcatcaccgatttggttatcaaccactgttctacagaacacgaatggttcaaagagagcagatcctcgaagaccaatccgaagcgtgactggttcttctggagacctcctaagggttatgacgccgaaggcaagccaattcctccaaacaattggaaatctttctttggtggttcagcttggacttttgatgaaactacaaatgaattttacctccgtttgtttgcgagtcgtcaagttgacttgaattgggagaatgaagactgcagaagggcaatctttgaaagtgctgttggattttggctggaccatggtgtagatggttttagaatcgataccgctggtttgtattcgaaacgtcctggtttaccagattccccaatttttgacaaaacctcgaaattacaacatccaaattgggggtctcacaatggtcctaggattcatgaatatcatcaagaactacacagatttatgaaaaacagggtgaaagatggtagagaaataatgacagtcggtgaagttgcccatggaagtgataatgctttatacaccagtgcagctagatacgaagtcagcgaagttttctccttcacgcacgttgaagttggtacctcgccatttttccgttataacatagtgcccttcaccttgaaacaatggaaagaagccattgcatcgaactttttgttcattaacggtactgatagttgggctaccacctacatcgagaatcacgatcaagcccggtcaattacgagatttgctgacgattcgccaaagtaccgtaaaatatctggtaagctgttaacattgctagaatgttcattgacaggtacgttgtatgtctatcaaggtcaggagataggccagatcaatttcaaggaatggcctattgaaaagtatgaggacgttgatgtgaaaaacaactacgagattatcaaaaaaagttttggtaaaaactcgaaggaaatgaaggatttttttaaaggaatcgccctactttctagagatcattcgagaactcccatgccatggacgaaagataagcccaatgctggatttactggcccagatgttaaaccttggtttttcttgaatgaatctttcgagcaaggaatcaatgttgagcaggaatccagagatgatgactcagttctcaatttttggaaaagggccttgcaagccagaaagaaatataaggaacttatgatttatggttacgatttccaattcattgatttagacagtgaccagatctttagcttcactaaagagtacgaagacaagacgctgtttgctgctttaaatttcagtggcgaagaaattgaattcagcctcccaagagaaggtgcttctttatcttttattcttggaaattatgatgatactgacgtttcctccagagttttgaaaccatgggaaggtagaatctacctcgtcaaataaaattagtgccggcttttttttagcgcgtactttaacgaaataacacatgatttttcacatgatttttgttagataaattttttatatgtaaatgatgatagcgtaaaagcactgttgataatttgtttcaccattatgggtaaatgtgtttttctacatgaccctcgttcattatgatatttagcgtgtatataaatgtgaattccaaattattaatgaggcataagaagcactatcctttctcttcggatgaaaacaagggagaagaaacctgtgctggtattaatgctgaaatgtcttgctaagaatcatacaaggtggtagttttatttaataaagaaaagaaaaggactagatataaaaagtgaaatgaatataagatagcgttaagagatgtccgcagtacttgacacataatttagcgttttctcgggaagctctgtgattttatgattcaataacacagcgtaattgatttcgtgatagttcgatcctatatgtaatctcacgtaacactcaggcgagttacaaaatcgattcaacattgccggcttatgcgtttacgtcaagtctgagcatgcctacccccttccgaacccgccttttattgtctagccttcagatgaactaaaccaatcatctgtccataattcctctgctttagacagtgttattaagcaaaagaaaataagcgcataagattcttgctacttcagtaactccacaacattaacaccccacaatcaatatctaaaagccaatgaag

SEQ ID NO: 2 - DNA, 9216 bp, synthetic construct
  misc_feature 1..9216: note = Vector sequence with MAL1 gene cluster from Cen.PK strain 113-7D
  source 1..9216: mol_type = other DNA
acaggaacgattgtcttgataatatgtgaaaagtgcacacgaaattagagggtgtcctttacaagtattcttagaaacacattcaagagcacaaaagtcgatgctttaagggtcaaggtggtggaaaacttgactggaattcttgacgaaaaaacaagaaaaacgtgattcgagcaatcataaacatacagccccgttccaaccggatcttgaggtttcccattttagatggaaataagcagagcaaaataaaaatcttgaacaagtaatagtggtgactgcaggttacgttggcatataaagtccgggtgacctgggtttcctgcaccaccagcccccatatgctagcacaatgggttttctttatccccggtcataattactcattttgctatattcttcataacttaagtacgcagatagagaaaattaataatctcgatatatattaaagtaaatgaaaagtagaaaatttagccagaactcttttttgcttcgagtatgactttaactaagcaaacatgcgccaagcaggcatgcgactgctgtcgtattcgtcgagtgaaatgcgatggtaaaaggccgtgtagcagttgcctacagaatagtttggattgcacttatctgcaaccgtcgagaaaaagaggtccgaagtccattaggttgaggagcttgaaaagaatagcagaagtgcagagggaaagcggtcctaacaccattgcaactgctcctgtaatatataagagggttcccaaaaagctaatcgatcagtgcttgcggctctatcacgataatttatacgtaatctggccccttctttcgtacgatgaccttcacaaacttctggaggaaaaatacaatgacaattacgtatattggtttctgaccgctttatcagcggccaccctcagtgatttacaaactgaaataaaatctgaagaggaagtcactttcacgggaaaacagttatctaatctttgcatctcatcgtgtcagcaatttgacgatttggataacagcaatatattcaatattatgacgtactactgtttgcatcgtagctttgcacaaatatcgaacgcaagaacttcttacagactctgttgtgaagcggtcggtctgattacggtagcagggttacatcgggaagaaacttacggatcccttacatttgaagaacagcaacttagacggaaactttattacttgcttctcatgacggagagatactatgccatatatcttcattgtgcgacgagcctggatgccacaatagcaccaccgcaacttgaacttgtaactgatcctcagctttctatggacagtttccttgaaatgattagggtatttactgtaccaggaaaatgtttcttcgatgctttagccgctgactctacagatgcttcttgcactgaagagtcattgaaaaagatatggaacgaactccacacaacttcctcggaaatagagccatggtctaacggttacatagacatctcattttcccggcattggattaggatactagcatggaagctagcttatcaaatgaggggtagcaacttttcattgaacgctaacaatgggcaaataccaatagaaattgcgagagatatgttaatagacacttacttaaccccagagaatctttacgatgtccatggtcccggggtaccagtgaaaacattagaaatagctactgctttggtggacattgtaggccagtatgatcataacatgaaattagaagcatggaatgttttgcatgatgtatgcaaatttgctttttctttaaaccactataacaatgatatgctgaagagattttccaccaaatgccagaatgccctaattactctgcccatttctaaacctttacaattggatggttatcccaaggataatgaagacatagacccttgattaattttcatttttgtgcatctcaacttcctggtaagtgatagctttccattgtagaaactgtgtttccgcaacacaagggtaaaattcactgctaattgcgacccattttcatgaacagagtaattaattttctatttggaggtctacttttacaagtataagactgcttcttaccatgatgtctccctattgaaaattatatttaataaaatacttttaggcacgctaacgttagcattcttcccagaattcctatactaacagttttcagtatatatacacttttttactgagtgctaagagccagattggatgagatgattgtgtactgatggagaattaacggttggagagctattactcacataacttcgtataatgtatgctatacgaagttatttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccaataacttcgtataatgtatgctatacgaagttatgagtggtataacaagacctgcaagtgtatggacatttaaagtaacagttaattgagaatacggttgacctggcatgttgttcgaatcaatatccaggcacaagtaccaggtgctaaagaaaaagtactctcatatttgcttgattgctgcttgggctattttaactaactactaacaatattttgcttaaaaaatggtaaatatgaatgttttacagaaaaataaaaaatgtatatatataaaatctcgagctagctgagggttttgggagcagtcaaagggattccttatttcttccaaaaaaaaaaaaacaacccttttacttaacatttatcagctgcatttaattctcgctgttttatgcttgaggactgactgatactctcatcagctagcgaatcatgttgagtttttccctttccgaatggatcaaccacagtagatgcaaattttctggcaggaaccccttggttgaaaagttcattaatttcactgaaggttctaccagttgtctcaggcagatcgatgatgacccaagctaaagtgactgctgtgaaaccaccccagtatagaccagttttggcaccccagttccaatcgctcacgtttagcatatagggcgttaatatagcgttaataacggccatgagattgtagcaaatacgggccagcactatagtcttagttctcaactccgctgatggaatttcagcaacgatacagtaaacaactgcaccgataccagcattgtaaaagaatgataaagccagcaataaaccaccggcaccattactagcgctgcttccagaaccaaaacccattccaccaataataaataagcagaccatttgaaatgcaagaccataggtcagtattgtccatctaccaacacggccagatattacccaggagcaaagtgtacccgctaacccaagacagtactgaattagagaaaaagtaaacgccttgtcggtggccatacctgctctttcaaaaaaatatgtcgagtaaccaagtaaaacggcaccgctactattttgagctacccaagttaaacatgcaagtctcgttcttcttccattaactcccttgaaacaattaaagaatgatcctgatttagatgctaaaagtctttctttttcaatagtcaattcaatctgctttaaagtaagatcaacttgaatgtccttctcggcgcctttaccactcaaaattctgcttaaagattttcttgcctcagcgaccctatcctttctcaccaaccaccagggcgactcaggagcgaaaaagataccgatcattaaaggagcaggccaaatccattgtaaagcaaatggcaatttatagcccaagtcggagttccctaaattctcttgtgagtttttcataataccagaggcgaagatttgaccaaataaccaacaaatgttggagtaactggtcatgtaatatcttaatgctaaagggcaaacttccgaagcataagtaacagccaaactttggaaacaaccccatggtatagctgagagaatttgtcccacagcaatcatagctaaacttttacagtagtagaggataaagatataagcagttaacaaaccaagtgctgtaatcatcgtataacgattccccataaattcaaccatataagtcgtgatttgcaaaccaatcatctcaccacaaaggacacacatgtttaaaccaatctgccattgggaagtaatttcgtaagaaccctccccgttcaaagtaccgaattttctctgaaaaactggcagggcatacagtgcgctcagtagtgcggtatcataaccttccataaccagggtagtagacactaatatggaccacagggctgcttttggatattttagcaacgcctgcttcaaagtcatgcttttttcctcgctgttagcttcatttgcatcatcagtagcgttcatctcattaatcacattctcgttatcttcgtcagaatctcctaactgggctgaattggtggtgaactctaagtggtctagctcaaaggcactatcctttttcccttcttcaaaatcttcagtattgaaaacctcctgttggtttacaatatctcttgaagactcagaaatgtttttatcctcattttttgaggcagccttcttcttgcttaccaatgaaatgatatttttcatattatactatttttttagttgtttgatgttcttctatgtagcatcagaaagaaacaccaacccgaaaattcttcaaacaatcaataccaaaccgctttatataaaaaattaagatgtcgacattccttattttttactgagttcgttaaagttgggtacactcttgattactgtaattgtctctgtatgtccctcaagcccggtacgttgtcattttctagtacgcatcaacggagtgttacatgatagatagaccgagtagaatctatggctatggggtaattaaaaccttaaagctcctttcgctgccatagtaatacgaatagaccttggctatagtaagttgcatctgtaccgtagagattcttgcaactcgcttaaactctcgcttttagataatatttctccttattgcgcgcttcgttgaaaatttcgctaaacacggggtttaagttaaagtttacaggatttatccggaaattttcgcggaccccacacaattaagaattggctcgaagagtgataacgcatacttttcttttctttttttagttcctagcgtacctaacgtaggtaacatgatttggatcgtgggatgatacaaacaacgtaagatgaatagttccttcctcaattcttcttgcagcatcattttcttgaggcgctctgggcaaggtataaaaagttccattaatacgtctctaaaaaattaaatcatccatctcttaagcagtttttttgataatctcaaatgtacatcagtcaagcgtaactaaattacataaatgactatttctgatcatccagaaacagaaccaaagtggtggaaagaggccacaatctatcaaatttacccagcaagttttaaagactccaataacgatggctggggtgatttaaaaggtatcacttccaagttgcagtatattaaagatcttggcgttgatgctatttgggtttgtccgttttatgactctcctcaacaagatatggggtatgatatatccaactacgaaaaggtctggcccacatatggtaccaatgaggactgttttgagctaattgacaagactcataagctgggtatgaaattcatcaccgatttggttatcaaccactgttctacagaacacgaatggttcaaagagagcagatcctcgaagaccaatccgaagcgtgactggttcttctggagacctcctaagggttatgacgccgaaggcaagccaattcctccaaacaattggaaatctttctttggtggttcagcttggacttttgatgaaactacaaatgaattttacctccgtttgtttgcgagtcgtcaagttgacttgaattgggagaatgaagactgcagaagggcaatctttgaaagtgctgttggattttggctggaccatggtgtagatggttttagaatcgataccgctggtttgtattcgaaacgtcctggtttaccagattccccaatttttgacaaaacctcgaaattacaacatccaaattgggggtctcacaatggtcctaggattcatgaatatcatcaagaactacacagatttatgaaaaacagggtgaaagatggtagagaaataatgacagtcggtgaagttgcccatggaagtgataatgctttatacaccagtgcagctagatacgaagtcagcgaagttttctccttcacgcacgttgaagttggtacctcgccatttttccgttataacatagtgcccttcaccttgaaacaatggaaagaagccattgcatcgaactttttgttcattaacggtactgatagttgggctaccacctacatcgagaatcacgatcaagcccggtcaattacgagatttgctgacgattcgccaaagtaccgtaaaatatctggtaagctgttaacattgctagaatgttcattgacaggtacgttgtatgtctatcaaggtcaggagataggccagatcaatttcaaggaatggcctattgaaaagtatgaggacgttgatgtgaaaaacaactacgagattatcaaaaaaagttttggtaaaaactcgaaggaaatgaaggatttttttaaaggaatcgccctactttctagagatcattcgagaactcccatgccatggacgaaagataagcccaatgctggatttactggcccagatgttaaaccttggtttctcttgaatgaatctttcgagcaaggaatcaatgttgagcaggaatccagagatgatgactcagttctcaatttttggaaaagggccttgcaagccagaaagaaatataaggaacttatgatttatggttacgatttccaattcattgatttagacagtgaccagatctttagcttcactaaagagtacgaagacaagacgctgtttgctgctttaaatttcagtggcgaagaaattgaattcagcctcccaagagaaggtgcttctttatcttttattcttggaaattatgatgatactgacgtttcctccagagttttgaaaccatgggaaggtagaatctacctcgtcaaataaaattagtgccggcttttttttagcgcgtactttaacgaaataacacatgatttttcacatgatttttgttagataaattttttatatgtaaatgatgatagcgtaaaagcactgttgataatttgtttcaccattatgggtaaatgtgtttttctacatgaccctcgttcattatgatatttagcgtgtatataaatgtgaattccaaattattaatgaggcataagaagcactatcctttctcttcggatgaaaacaagggagaagaaacctgtgctggtattaatgctgaaatgtcttgctaagaatcatacaaggtggtagttttatttaataaagaaaagaaaaggactagatataaaaagtgaaatgaatataagatagcgttaagagatgtccgcagtacttgacacataatttagcgttttctcgggaagctctgtgattttatgattcaataacacagcgtaattgatttcgtgatagttcgatcctatatg

SEQ ID NO: 3 - DNA, 3736 bp, synthetic construct
  misc_feature 1..3736: note = MAL2-8c construct
  source 1..3736: mol_type = other DNA
agaactttgactcttctacaacgtgaatgcctttgataagaatgaaattccaaaacaagtaatgttgggaggtagatttcctccactgctaattccaactacgtgtgcatttttcaatagtaatattccgtcacaagaggcttattttcattttctctaccctcatctttttctcacttttttccttacaatgaatacatgtgatatagatacttaattgtctgttttgcgagcttgcttcttcatatctatgtaatatgggccaggtcaacccaacatctaccaattatctatatgaagaaaaatatgattggtagttaccgccaatgcatagattttagacaacttaataaggccatgttaaagggtgcattcccactatcgcgcttaggattggatgaagcataacttttcttcactgtcaaattgcatcgtagttatatcagatccaaataaaaaatgaaaataacaataacaagccttctattttttcttgtcatgtttaaacggtcatggaagacctgaactaaagtgttttagtaaaccaattggagtgagagtttttcattccgaagattctttatctcaaaatttctttatcgaaagacacttctgtgtcactgtccgttcaatcagtcagatagttccaactccgatgtcttccaatacctcaacgaagaccgaaaaataaaaggtttgtttgacggagtgtgttgattagtgcattggtgacgtggggtagcaaaatccagatacttctattttttgaaaaagaaaggagagagtgctagaatgttttcacgtttatcagtacacgaaaaacaaaacctgaagcaaatgattaccataactattgtccacttatggggaagttgctaaaaataacacattatttactaagggaacacaatttgctcatagtatacttgactttttttacttaacttttgcagcgattggtgatgaaaatgagcatgcagactaataggtaggaaagtagaactacttagaaacattctccttaagtgttttcaccactaagcattttatatttaattgttaaaaaatatatactattgaagaaccactttcctgaaatatcaagaacaaaaaagtctgcactatggtcccgcaattgatgcatttgagaattcttttaactcaatagtaatatgcattgttcttatctaaaaaattgcaggtacctgcagactaatccgggtcatgatctgcgctgcgcccgtcatcccaccccgtgctgcctgccacttgaagctaccccgggtttaataattcgttctttaagttctacaacttaaatacaggcagctaaaaaactgggttcgagagttttccactttatagacaaaaataaaaatactgccagaaaatttatcatataataatatgggtattgcgaaacagtcttgcgactgctgtcgcgttcgtcgagtaaagtgtgacaggaataaaccatgtaatcgctgcactcagcgcaatttgaactgcacttatcttcaaccgttgaaaaagagaggtccaaaatccattagagcaggaagcttaaaaaaaatagcggaagtgcagatggtgagtatgaataataatattatgaccgctccggtggtatgtaagaaggttccgaaaaacctgattgatcaatgtttgaggttgtatcatgataacttatatgtaatttggccaatgctttcctacgatgatcttcacaagcttttggaggaaaattatgaggactgcagcacttattggtttctggtatccctttcggcagctactcttagcgacttgcaaattgaaatagagtatgaggaaggagtcacttttactggagagcagttatgcactctttgcatgttatctcggcaattctttgacgaccttagtaacagcgacatatttcgaatcatgacatactattgtttgcaccgttgttacgcgcagtttgctgatacaagaacttcatacagactttcttgtgaggctattggcctcatcaagatagctggattccatcgggaagaaacctatgaattccttcccttcggtgaacaacaactcagaaggaaagtttactatttacttcttatgacagagagattttacgctgtatatattaagtgtgtcacgagcctagatacaacaatagcgccaccactaccagaggttgtaacagaccctcgtctttctctggaaagcttccttgaggtgattagagttttcactgtacctggaaagtgtttttatgatgctttggctactaactgtgtcgatgattcctgcaccgaagactctctaaaaaggatatggaacgaacttcataccacatcacttgatatagagccatggtcttatggctatgtggacatttcattttctcgacattggattagggcgctggcttggaagctagtgtttcagatgaatggtaccaagtttttctcaaacgccaataatgctcacatattggtcgaaattgcaaaggatatgctggacgacatattcttaactccaaacaacctgtatgatgtacatggtcctggaataccaatgaaatcattggaagtagccaatgcattggtagatatcgtaaataagtatgatcacaatatgaagttggaggcttggaatattttgtgcgatgtatccaagttcgttttctccctgaaacattgcaatcataaaatgtttcaaaggttttcaactaaatgtcagagtgctctaatcgatttgcctatctctagaccactgcgcctaaatgatgattccaaagatgaagacgacataattccttaatttattgttcacgccgttcacttatacgagatagatatactgatagagtgtgagtgatattcttaagtcttgcttttcgagggtgtaagaagctatgttcttcaggcgagattattctactcctgccttacttgtttgtaatatttagttctgatggtcatgataattctatatacagttacattaagtatatacttaagcgggcagcttactaatataaattttgtggcatttttgttgggatatgagaatcatgtatcgttgatttacaaagcgaatttacgttaccaggaatagggaatactctcttgaattctaacataagcacagaaatgctgaaagaatacgtcaaaaagtaaatttacagaattaaaaaaaaaataattgttgccggaacatgaatagagtgtatcagtttaaacgcacactacttcataatggtgcaaatttgccctcattacgtgataacaccactctaactgatgctcgtaatgtgttaaagtacttacaagtgcttggttttccaagcaacaaaatagcggctgcggatactgttggaactcttatcatatttagcaatcgtgcggaagctaacagtaccgctatgacgaagacagtgtcatactgttatcgtaactacgggcatagtttttacttcactcattacaaatacgactattttcctagtgagattagttatatggcaaaacttggcgatgccgccgtcaaccatacggacttacctcactttaggaacaacaaacggctaacaacgcaagaagtcaatgccttccaacatccaattgtcgaattttagtaagtgctcaggtattacgttatgtacatgtatgatacttttgattaacatcctttatacacaaagatgtatgcatgaatggtgcaaatatctcgacgatgcgca

SEQ ID NO: 4 - protein, 616 aa, synthetic construct
  REGION 1..616: note = MAL11 product
MKNIISLVSKKKAASKNEDKNISESSRDIVNQQEVFNTEDFEEGKKDSAFELDHLEFTTNSAQLGDSDEDNENVINEMNATDDANEANSEEKSMTLKQALLKYPKAALWSILVSTTLVMEGYDTALLSALYALPVFQRKFGTLNGEGSYEITSQWQIGLNMCVLCGEMIGLQITTYMVEFMGNRYTMITALGLLTAYIFILYYCKSLAMIAVGQILSAIPWGCFQSLAVTYASEVCPLALRYYMTSYSNICWLFGQIFASGIMKNSQENLGNSDLGYKLPFALQWIWPAPLMIGIFFAPESPWWLVRKDRVAEARKSLSRILSGKGAEKDIQVDLTLKQIELTIEKERLLASKSGSFFNCFKGVNGRRTRLACLTWVAQNSSGAVLLGYSTYFFERAGMATDKAFTFSLIQYCLGLAGTLCSWVISGRVGRWTILTYGLAFQMVCLFIIGGMGFGSGSSASNGAGGLLLALSFFYNAGIGAVVYCIVAEIPSAELRTKTIVLARICYNLMAVINAILTPYMLNVSDWNWGAKTGLYWGGFTAVTLAWVIIDLPETTGRTFSEINELFNQGVPARKFASTVVDPFGKGKTQHDSLADESISQSSSIKQRELNAADKC

SEQ ID NO: 5 - protein, 584 aa, synthetic construct
  REGION 1..584: note = MAL12 product
MTISDHPETEPKWWKEATIYQIYPASFKDSNNDGWGDLKGITSKLQYIKDLGVDAIWVCPFYDSPQQDMGYDISNYEKVWPTYGTNEDCFELIDKTHKLGMKFITDLVINHCSTEHEWFKESRSSKTNPKRDWFFWRPPKGYDAEGKPIPPNNWKSFFGGSAWTFDETTNEFYLRLFASRQVDLNWENEDCRRAIFESAVGFWLDHGVDGFRIDTAGLYSKRPGLPDSPIFDKTSKLQHPNWGSHNGPRIHEYHQELHRFMKNRVKDGREIMTVGEVAHGSDNALYTSAARYEVSEVFSFTHVEVGTSPFFRYNIVPFTLKQWKEAIASNFLFINGTDSWATTYIENHDQARSITRFADDSPKYRKISGKLLTLLECSLTGTLYVYQGQEIGQINFKEWPIEKYEDVDVKNNYEIIKKSFGKNSKEMKDFFKGIALLSRDHSRTPMPWTKDKPNAGFTGPDVKPWFLLNESFEQGINVEQESRDDDSVLNFWKRALQARKKYKELMIYGYDFQFIDLDSDQIFSFTKEYEDKTLFAALNFSGEEIEFSLPREGASLSFILGNYDDTDVSSRVLKPWEGRIYLVK

SEQ ID NO: 6 - protein, 473 aa, synthetic construct
  REGION 1..473: note = MAL13 product
MTLTKQTCAKQACDCCRIRRVKCDGKRPCSSCLQNSLDCTYLQPSRKRGPKSIRLRSLKRIAEVQRESGPNTIATAPVIYKRVPKKLIDQCLRLYHDNLYVIWPLLSYDDLHKLLEEKYNDNYVYWFLTALSAATLSDLQTEIKSEEEVTFTGKQLSNLCISSCQQFDDLDNSNIFNIMTYYCLHRSFAQISNARTSYRLCCEAVGLITVAGLHREETYGSLTFEEQQLRRKLYYLLLMTERYYAIYLHCATSLDATIAPPQLELVTDPQLSMDSFLEMIRVFTVPGKCFFDALAADSTDASCTEESLKKIWNELHTTSSEIEPWSNGYIDISFSRHWIRILAWKLAYQMRGSNFSLNANNGQIPIEIARDMLIDTYLTPENLYDVHGPGVPVKTLEIATALVDIVGQYDHNMKLEAWNVLHDVCKFAFSLNHYNNDMLKRFSTKCQNALITLPISKPLQLDGYPKDNEDIDP